(19) United States
(12) Patent Application Publication
(10) Pub. No.: US 2025/0154251 A1
(43) Pub. Date: May 15, 2025

(54) COMPANION DIAGNOSTIC FOR HUMAN CEACAM1 DIRECTED THERAPEUTIC AGENTS

(71) Applicant: THE BRIGHAM AND WOMEN'S HOSPITAL, INC., Boston, MA (US)

(72) Inventors: Richard S. BLUMBERG, Boston, MA (US); Yu-Hwa HUANG, Boston, MA (US); Amit GANDHI, Boston, MA (US)

(73) Assignee: THE BRIGHAM AND WOMEN'S HOSPITAL, INC., Boston, MA (US)

(21) Appl. No.: 18/835,573

(22) PCT Filed: Feb. 3, 2023
(86) PCT No.: PCT/US2023/061931

§ 371 Date: Aug. 2, 2024

Related U.S. Application Data
(60) Provisional application No. 63/306,147, filed on Feb. 3, 2022.

(51) Int. Cl. C07K 16/28

(52) U.S. Cl. CPC C07K 16/2803; C07K 2317/24; C07K 2317/33

(57) ABSTRACT

Provided herein are methods and compositions for determining if a subject diagnosed with a CEACAM1-related disease or disorder is administered a CEACAM1 antibody or antigen-binding agent as a treatment.

### CROSS REFERENCE TO RELATED APPLICATIONS

This application claims benefit under 35 U.S.C. § 119(e) to U.S. Provisional Application No. 63/306,147 filed Feb. 3, 2022, the contents of which are incorporated herein by reference in its entirety.

### GOVERNMENT SUPPORT

This invention was made with government support under Grant No. DK51362 awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Feb. 2, 2023, is named “043214-191430USPX_SL.xml” and is 23,259 bytes in size.

### TECHNICAL FIELD

The technology described herein relates to the field of molecular biology and medicine. More specifically, the technology relates to the determination of appropriate therapy to treat subjects with CEACAM1-related diseases or disorders.

### BACKGROUND

Carcinoembryonic antigen-related cell adhesion molecule 1 (CEACAM1) is a member of the carcinoembryonic antigen (CEA) family of immunoglobulin (Ig) like transmembrane glycoproteins. CEACAM family members are involved in cell-cell recognition and modulate cellular processes that range from the shaping of tissue architecture and neovascularization to the regulation of insulin homeostasis and T cell proliferation. The CEACAM family includes CEACAM1, CEACAM3, CEACAM5, CEACAM6, CEACAM7, CEACAM8, CEACAM16, CEACAM18, CEACAM19, CEACAM20, and CEACAM21.

The immune system protects the body from foreign invaders and diseased cells; but immune disorders, particularly those associated with T-cell tolerance, such as cancers, can wreak havoc. According to the most recent data from the World Health Organization, ten million people around the world were diagnosed with the cancer in 2000, and six million died from it. Moreover, statistics indicate that the cancer incidence rate is on the rise around the globe. In America, for example, projections suggest that fifty percent of those alive today will be diagnosed with some form of cancer at some point in their lives.

Modern technology, such as that involving the use of hybridomas, has made available a source of highly specific and potent monoclonal antibodies useful in general diagnostic and clinical procedures.

### SUMMARY

A disease or disorder in which CEACAM1 plays a role in the pathology can be treated with an inhibitor of CEACAM1. Antibodies or constructs including antigen-binding domains, portions or fragments thereof that bind to CEACAM1 can be used as inhibitors of the protein's function. However, allelic variation in CEACAM1 that influences the epitopes displayed by a given CEACAM1 polypeptide renders some antibodies more effective than others. The technology described herein relates to methods and compositions for determining therapy for a subject diagnosed with a CEACAM1-related disease or disorder by detecting the subject's genotype and then determining if an antibody or antigen-binding agent that binds a given face or domain of CEACAM1 is to be administered.

In one aspect, described herein is a method comprising: receiving results of an hCEACAM1 genotyping assay performed on a sample from a subject in need of therapy for a CEACAM1-related disease or disorder; and selecting therapy with an agent comprising an antibody or antigen-binding domain that binds an epitope in the GF-CC′ domain of CEACAM1 when the subject's hCEACAM1 genotype is homozygous wild-type with regard to A49, Q89, Q44 and Y34 or heterozygous for A49V, Q89H, Q44L and/or Y34C, and selecting therapy with an agent other than an agent comprising an antibody or antigen-binding domain that binds an epitope in the GF-CC′ domain of CEACAM1 when the subject's hCEACAM1 genotype is homozygous for A49V, Q89H, Q44L, and/or Y34C hCEACAM1 alleles.

In another aspect is a method comprising: genotyping a sample from a subject in need of therapy for a CEACAM1-related disease or disorder to detect the subject's hCEACAM1 genotype; and selecting therapy with an agent comprising an antibody or antigen-binding domain that binds an epitope in the GF-CC′ domain of CEACAM1 when the subject's hCEACAM1 genotype is homozygous wild-type with regard to A49, Q89, Q44 and Y34 or heterozygous for A49V, Q89H, Q44L and/or Y34C, and selecting therapy with an agent other than an agent comprising an antibody or antigen-binding domain that binds an epitope in the GF-CC′ domain of CEACAM1 when the subject's hCEACAM1 genotype is homozygous for A49V, Q89H, Q44L, and/or Y34C hCEACAM1 alleles.

In one embodiment of this aspect and all other aspects provided herein, the subject has or has been diagnosed with a chronic disease.

In one embodiment of this aspect and all other aspects provided herein, the chronic disease is selected from the group consisting of: cancer; a persistent infection; and a chronic viral infection.

In one embodiment of this aspect and all other aspects provided herein, the subject is a human.

In one embodiment of this aspect and all other aspects provided herein, the antibody or antigen-binding domain that binds an epitope of the GF-CC′ domain of CEACAM1 comprises a heavy chain variable region and a light chain variable region; each of the heavy chain and the light chain variable regions comprises a CDR1, CDR2, and CDR3; and the sequence of CDR1H comprises the sequence X1HX2X3S (SEQ ID NO: 1); Xi is A, D, N, or S; X2 is A or G; and X3 is an amino acid with a hydrophobic side chain including I or M; the sequence of CDR2H comprises the sequence TISSGGTYTYYPDSVKG (SEQ ID NO:2); the sequence of CDR3H comprises the sequence HX4X5DYX6PX7WFAX8 (SEQ ID NO:3); X4 is D, G, or P; X5 is F or P; Xe is D or F; X7 is A or Y; and Xs is L, H, or F; the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX9X10X11X12PX13T (SEQ ID NO:6); X9 is W or N; X10 is S or T; Xu is A or an amino acid with a neutral hydrophilic side chain including S, N, and T; X12 is L, F, or N; and X13 is P or F.

In one embodiment of this aspect and all other aspects provided herein, the sample is selected from a blood sample, a tissue sample, a tumor sample, and a cell sample.

In one embodiment of this aspect and all other aspects provided herein, the genotyping assay comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

In one embodiment of this aspect and all other aspects provided herein, the method further comprises administering the selected therapy.

In one embodiment of this aspect and all other aspects provided herein, the method further comprises, when the CEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and adjusting amount and/or dosing of the agent to be administered as a function of the degree of binding of the agent to the subject's CEACAM1.

In one embodiment of this aspect and all other aspects provided herein, the assaying comprises a method selected from flow cytometry, mass cytometry, immunohistochemistry, immunofluorescence or FRET assay, surface plasmon resonance, radioimmunoassay, and bio-layer interferometry.

In one embodiment of this aspect and all other aspects provided herein, the method further comprises, before the genotyping, the step of extracting genomic DNA from a sample obtained from the subject.

In another aspect as described herein is a method of treating a subject in need thereof for a CEACAM1-related disease or disorder, the method comprising: genotyping a sample from the subject to detect the subject's hCEACAM1 genotype; and when the subject's hCEACAM1 genotype is homozygous wild-type with regard to A49, Q89, Q44 and Y34, administering a therapeutically effective amount of an agent comprising an antibody or antigen-binding domain that binds an epitope in the GF-CC′ domain of CEACAM1; and when the subject's hCEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and administering a therapeutically effective amount of the agent, wherein the amount or dosing schedule of the agent administered is adjusted as a function of the degree of binding of the agent to the subject's CEACAM1; and when the subject's hCEACAM1 genotype is homozygous for A49V, Q89H, Q44L and/or Y34C, administering a therapeutically effective amount of a therapy with an agent other than one comprising an antibody or antigen-binding domain that binds an epitope in the GF-CC′ domain of CEACAM1, whereby the CEACAM1-related disease or disorder is treated.

In one embodiment of this aspect and all other aspects provided herein, the subject has or has been diagnosed with a chronic disease.

In one embodiment of this aspect and all other aspects provided herein, the chronic disease is selected from the group consisting of: cancer; a persistent infection; and a chronic viral infection.

In one embodiment of this aspect and all other aspects provided herein, the subject is a human.

In one embodiment of this aspect and all other aspects provided herein, the antibody or antigen-binding domain that binds an epitope of the GF-CC′ domain of CEACAM1 comprises a heavy chain variable region and a light chain variable region; each of the heavy chain and the light chain variable regions comprises a CDR1, CDR2, and CDR3; and the sequence of CDR1H comprises the sequence X1HX2X3S (SEQ ID NO: 1); Xi is A, D, N, or S; X2 is A or G; and X3 is an amino acid with a hydrophobic side chain including I or M; the sequence of CDR2H comprises the sequence TISSGGTYTYYPDSVKG (SEQ ID NO:2); the sequence of CDR3H comprises the sequence HX4X5DYX6PX7WFAX8 (SEQ ID NO:3); X4 is D, G, or P; X5 is F or P; Xe is D or F; X7 is A or Y; and Xs is L, H, or F; the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX9X10X11X12PX13T (SEQ ID NO:6); X9 is W or N; X10 is S or T; Xu is A or an amino acid with a neutral hydrophilic side chain including S, N, and T; X12 is L, F, or N; and X13 is P or F.

In one embodiment of this aspect and all other aspects provided herein, the sample is selected from a blood sample, a tissue sample, a tumor sample, and a cell sample.

In one embodiment of this aspect and all other aspects provided herein, the genotyping assay comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

In one embodiment of this aspect and all other aspects provided herein, assaying a sample from the subject comprising CEACAM1 to determine the degree to which the subject's CEACAM1 is bound by the agent comprises a method selected from flow cytometry, mass cytometry, immunohistochemistry, immunofluorescence or FRET assay, surface plasmon resonance, radioimmunoassay, and bio-layer interferometry.

In another aspect as described herein is an assay comprising: i) extracting genomic DNA from a biological sample from a subject in need of therapy for a CEACAM1-related disease or disorder; ii) genotyping the subject's DNA with regard to sequence corresponding to CEACAM A49, Q89, Q44 and Y34 to thereby determine whether the subject is homozygous wild-type for A49, Q89, Q44 and Y34 or homozygous or heterozygous for each of A49V, Q89H, Q44L and Y34C.

In one embodiment of this aspect and all other aspects provided herein, the CEACAM1-related disease or disorder is selected from the group consisting of: cancer; a persistent infection; and a chronic viral infection.

In one embodiment of this aspect and all other aspects provided herein, the subject is a human.

In one embodiment of this aspect and all other aspects provided herein, the biological sample is selected from a blood sample, a tissue sample, a tumor sample, and a cell sample.

In one embodiment of this aspect and all other aspects provided herein, the genotyping comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

In one embodiment of this aspect and all other aspects provided herein, the assay further comprising iii) assaying the subject's CEACAM1 protein to determine the degree to which the subject's CEACAM1 is bound by an antibody or antigen-binding domain that binds an epitope in the GF-CC′ domain of CEACAM1.

In one embodiment of this aspect and all other aspects provided herein, the assaying of step (iii) comprises a method selected from flow cytometry, mass cytometry, immunohistochemistry, immunofluorescence or FRET assay, surface plasmon resonance, radioimmunoassay, and bio-layer interferometry.

In another aspect as described herein is an assay comprising: i) extracting proteins comprising CEACAM1 from a biological sample from a subject in need of therapy for a CEACAM1-related disease or disorder; and ii) assaying the subject's CEACAM1 in the extracted proteins to determine the degree to which the subject's CEACAM1 is bound by an antibody or antigen-binding domain that binds an epitope in the GF-CC′ domain of CEACAM1.

In one embodiment of this aspect and all other aspects provided herein, the assaying of step (ii) comprises a method selected from flow cytometry, mass cytometry, immunohistochemistry, immunofluorescence or FRET assay, surface plasmon resonance, radioimmunoassay, and bio-layer interferometry.

In one embodiment of this aspect and all other aspects provided herein, the assay further comprises iii) genotyping the subject's DNA with regard to sequence corresponding to CEACAM A49, Q89, Q44 and Y34 to thereby determine whether the subject is homozygous wild-type for A49, Q89, Q44 and Y34 or homozygous or heterozygous for each of A49V, Q89H, Q44L and Y34C.

In one embodiment of this aspect and all other aspects provided herein, the genotyping comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

In another aspect as described herein is a method of selecting a hCEACAM1 antibody therapy for a subject in need thereof, comprising: analyzing genomic DNA from a biological sample taken from the subject for the subject's genotype for alleles of hCEACAM1 at sites corresponding to A49, Q89, Q44 and Y34, and selecting therapy with an agent comprising an hCEACAM1 antibody or antigen-binding fragment thereof that binds an epitope in the GF-CC′ domain of CEACAM1 when the genotype is found to be homozygous for A49, Q89, Q44 and Y34, or heterozygous for one or more of A49V, Q89H, Q44L, and Y34C alleles for CEACAM1; or selecting therapy with an agent other than the agent comprising an hCEACAM1 antibody or antigen-binding fragment thereof that binds an epitope in the GF-CC′ domain of CEACAM1 when the genotype is found to be homozygous for one or more of A49V, Q89H, Q44L, and Y34C alleles for CEACAM1.

In one embodiment of this aspect and all other aspects provided herein, the subject has or has been diagnosed with a chronic disease.

In one embodiment of this aspect and all other aspects provided herein, the chronic disease is selected from the group consisting of: cancer; a persistent infection; and a chronic viral infection.

In one embodiment of this aspect and all other aspects provided herein, the subject is a human.

In one embodiment of this aspect and all other aspects provided herein, the antibody or antigen-binding domain that binds an epitope of the GF-CC′ domain of CEACAM1 comprises a heavy chain variable region and a light chain variable region; each of the heavy chain and the light chain variable regions comprises a CDR1, CDR2, and CDR3; and the sequence of CDR1H comprises the sequence X₁HX₂X₃S (SEQ ID NO: 1); X₁ is A, D, N, or S; X₂ is A or G; and X₃ is an amino acid with a hydrophobic side chain including I or M; the sequence of CDR2H comprises the sequence TISSGGTYTYYPDSVKG (SEQ ID NO:2); the sequence of CDR3H comprises the sequence HX₄X₅DYX₆PX₇WFAX₈ (SEQ ID NO:3); X₄ is D, G, or P; X₅ is F or P; X₆ is D or F; X₇ is A or Y; and X₈ is L, H, or F; the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX₉X₁₀X₁₁X₁₂PX₁₃T (SEQ ID NO:6); wherein X₉ is W or N; X₁₀ is S or T; X₁₁ is A or an amino acid with a neutral hydrophilic side chain including S, N, and T; X₁₂ is L, F, or N; and X₁₃ is P or F.

In one embodiment of this aspect and all other aspects provided herein, the sample is selected from a blood sample, a tissue sample, a tumor sample and a cell sample.

In one embodiment of this aspect and all other aspects provided herein, the genotype analysis comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

In one embodiment of this aspect and all other aspects provided herein, the method further comprising administering the selected therapy.

In one embodiment of this aspect and all other aspects provided herein, the method further comprising, when the CEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and adjusting amount and/or dosing of the agent to be administered as a function of the degree of binding of the agent to the subject's CEACAM1.

In one embodiment of this aspect and all other aspects provided herein, the assaying comprises a method selected from flow cytometry, mass cytometry, immunohistochemistry, immunofluorescence or FRETassay, surface plasmon resonance, radioimmunoassay, and bio-layer interferometry.

### Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein, “reducing pathology” refers to the reduction of one or more symptoms or markers of a CEACAM1-related disease or disorder by at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95% or more.

As used herein, a “CEACAM1-related disease or disorder” is a disease or disorder in which CEACAM1-mediated signaling contributes to disease pathology, for example, CEACAM1 expression within a pathologic lesion such as within a cancer and the tumor cells themselves, and in which that pathology can be effectively treated by inhibiting CEACAM1 function, including inhibiting interactions of CEACAM1 with one or more CEACAM1 ligands. Symptoms of a CEACAM1-related disease or disorder include altered infiltration pattern of immune cells in the microenvironment, unresponsive T cells, low levels of T cells, or an elevated level or increase in regulatory T cells relative to an appropriate reference or control.

The terms “decrease”, “reduced”, “reduction”, or “inhibit” are all used herein to mean a decrease or lessening of a property, level, or other parameter (such as the amount or relative level of a disease symptom) by a statistically significant amount. In some embodiments, “reduce,” “reduction” or “decrease” or “inhibit” typically means a decrease in a property, level or other parameter by at least 10% as compared to a reference and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. Rather, the term “complete inhibition” is used to refer to a 100% inhibition as compared to an appropriate reference level. A decrease in a symptom of a given cancer can be preferably down to a level accepted as within the range of normal for an individual without the cancer.

The terms “increased,” “increase” or “enhance” or “activate” are all used herein to generally mean an increase of a property, level, or other parameter (e.g., amount or relative level of a disease parameter, marker or symptom) by a statistically significant amount; for the avoidance of any doubt, the terms “increased”, “increase” or “enhance” or “activate” means an increase in the level of a parameter, marker or symptom by at least 10% as compared to a reference level (e.g., the level in one or more healthy individuals), for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, an “antibody agent” is a therapeutic composition comprising an anti-CEACAM1 antibody or antigen-binding portion or fragment thereof. The antibody or antigen-binding portion or fragment thereof in an antibody agent can be configured in any of a number of formats, including, without limitation, native antibodies, humanized or chimeric antibodies, scFv, bi- or multispecific antibodies, dAb, Fab, Fab′, F(ab′)2, Fd, Fd′, diabodies, or linear antibodies, among others.

As used herein, a “CP08-based antibody agent” includes an antibody agent in which the antigen-binding domain has a heavy chain variable region and a light chain variable region, each of the heavy chain and the light chain variable regions comprises a CDR1, CDR2, and CDR3; and the sequence of CDR1H comprises the sequence X₁HX₂X₃S (SEQ ID NO: 1); X₁ is A, D, N, or S; X₂ is A or G; and X₃ is an amino acid with a hydrophobic side chain including I or M; the sequence of CDR2H comprises the sequence TISSGGTYTYYPDSVKG (SEQ ID NO:2); the sequence of CDR3H comprises the sequence HX₄X₅DYX₆PX₇WFAX₈ (SEQ ID NO:3); X₄ is D, G, or P; X₅ is F or P; X₆ is D or F; X₇ is A or Y; and X₈ is L, H, or F; the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX₉X₁₀X₁₁X₁₂PX₁₃T (SEQ ID NO:6); wherein X₉ is W or N; X₁₀ is S or T; X₁₁ is A or an amino acid with a neutral hydrophilic side chain including S, N, and T; X₁₂ is L, F, or N; and X₁₃ is P or F. In one embodiment, a CP08-based antibody agent has CDRs of SEQ ID Nos 1, 2, 3, 4, 5, 6 or an affinity-matured version thereof.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### BRIEF DESCRIPTION OF THE DRAWINGS

FIG. 1 shows a crystal structure illustrating the CEACAM1:CEACAM1 homodimer interface (PDB ID: 4QXW). One CEACAM1 monomer is shown in on the left, the other one on the right. Residues Y34, Q44, Q89, and N97 form a YQQN pocket.

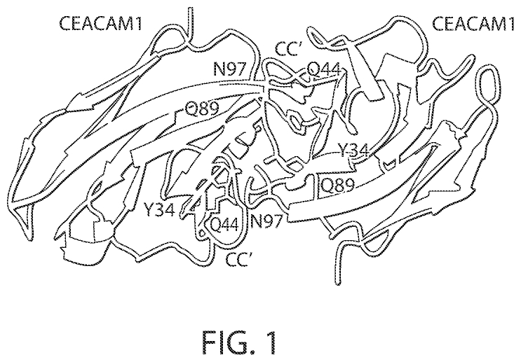

### DETAILED DESCRIPTION

Allelic variation in the CEACAM1 polypeptide renders some CEACAM1-related diseases less susceptible to treatment with certain CEACAM1 inhibitors. The technology described herein relates to methods for determining whether a given subject diagnosed with a CEACAM1-related disease or disorder is a candidate for therapy with a given anti-CEACAM1 inhibitor. In particular, allelic variation that affects the surface of CEACAM1 known as the GF-CC′ face (also referred to as the FG-CC′ face) influences whether antibodies that interact with this face will be therapeutically effective. The methods described herein relate generally to methods in which a subject's genotype in regard to CEACAM1 is determined, and that genotype informs whether an antibody or antigen-binding agent that interacts with the GF-CC′ face is likely to be effective in treating the subject's CEACAM1-related disease or disorder. Decisions for whether to administer a given antibody or antigen-binding agent that interacts with the GF-CC′ face of CEACAM1, or to administer a relatively higher or lower dose of such agent can be informed by the genotype, which can be homozygous wild-type, heterozygous for one or more allelic variants, or homozygous for one or more allelic variants affecting the GF-CC′ face of CEACAM1. If a subject is homozygous for one or more allelic variations that affect the GF-CC′ face, it is unlikely that an antibody or antigen-binding fragment that binds to the wild-type GF-CC′ face will be effective. If, on the other hand, the subject is homozygous wild-type or heterozygous for one or more alleles affecting the GF-CC′ face, it is more likely that an antibody or antigen-binding fragment that binds the wild-type face will be effective, e.g, at a first dose or frequency for homozygous wild-type or at a second, higher dose for heterozygous individuals.

The following describes considerations and methodology that facilitate one of ordinary skill in the art to put the technology disclosed herein into practice.

### CEACAM1

Carcinoembryonic antigen-related cell adhesion molecule 1 (CEACAM1) is a human transmembrane glycoprotein and a member of the carcinoembryonic antigen (CEA) gene family, which is a member of the immunoglobulin superfamily. It is also known as Cluster of Differentiation 66a (CD66a). CEACAM1 family members are involved in cell-cell recognition and modulate cellular processes that range from the shaping of tissue architecture and neovascularization to the regulation of insulin homeostasis and T cell proliferation.

Various cellular activities have been attributed to the CEACAM1 protein, including roles in the differentiation and arrangement of tissue three-dimensional structure, angiogenesis, apoptosis, tumor suppression, metastasis, and the modulation of innate and adaptive immune response. Cell types that express CEACAM1, include certain tumor cells, T cells, natural killer (NK) cells, and certain macrophages.

For instance, high CEACAM1 expression occurs in a variety of cancers such as melanoma, colorectal, gastric, pancreatic, bladder, and thyroid cancer and is associated with worse tumor progression, metastasis, and poor clinical prognosis. Non-small cell lung cancers (NSCLC), for example, with high CEACAM1 expression exhibit high microvessel density, distant metastases, and shorter median overall survival and progression free survival. CEACAM1 expression has also been strongly correlated with distant metastasis of pancreatic adenocarcinoma. CEACAM1 expression on tumors promotes CEACAM1-mediated inhibition of T and NK cells. Consequently, inhibiting CEACAM1 activity can inhibit tumor cell metastasis and the formation of a cancer stem cell niche.

CEACAM1 is also expressed in certain immune system cells and plays a role in immune suppression and immune cell exhaustion. High CEACAM1 expression on tumor infiltrating lymphocytes (TILs) and other tumor infiltrating immune cells from gastric, lung, melanoma, colorectal cancer and glioma, for example, is associated with a poor prognosis. On T cells, CEACAM1 expression is mostly excluded from resting (naïve) T cells, while the protein is expressed at high levels on activated T cells. CEACAM1-L is the dominant isoform expressed in most T cells and acts as an inhibitory receptor downregulating T cell activation and suppressing T cell functions. As such, inhibition of CEACAM1 on T-cells can recover T cell activity and increase anti-tumor responses.

CEACAM1 also mediates intercellular adhesion via the extracellular portion of CEACAM1 containing a IgV-like N-domain, which is involved in homophilic (CEACAM1:CEACAM1) and heterophilic interactions (e.g. with CEA, CEACAM5, CEACAM8, T cell-immunoglobulin and mucin-domain containing 3 (TIM-3) protein, Helicobacter pylori adhesin HopQ, Neisseria gonorrhoeae meningitidis opacity proteins (OPA), Moraxella sp. Opa-like protein O1pA, Haemophilus influenzae outer membrane protein (OMP) P1, Haemophilus aegyptius OMP P 1, Candida albicans, and influenza viruses such as H5N1). TIM-3 was identified as a Th1 specific cell surface protein that is expressed on activated T cells, subsets of dendritic cells and macrophages and NK cells. TIM-3 is an activation-induced inhibitory molecule that has been implicated in tolerance, and shown to induce T cell exhaustion in chronic viral infections and cancer. CEACAM1, which is also expressed on activated T cells, has been shown to interact with TIM-3, and this interaction is important for TIM-3-mediated T cell inhibition.

As indicated above, CEACAM1 also serves as cellular receptor on the apical membrane of mucosal cells for a variety of Gram-negative bacterial pathogens associated with the human mucosa, as well as with fungal pathogens such as Candida albicans. For instance, N. gonorrhoeae, N. meningitidis, Moraxella catarrhalis, H. influenza, H. aegyptius and pathogenic Escherichia coli strains possess well-characterized CEACAM1-binding adhesins. CEACAM1 engagement with bacterial adhesins triggers endocytosis of the bacteria into epithelial cells and transcytosis of microorganisms through intact epithelial layers, thus allowing the microorganisms to exploit CEACAM1 during mucosal colonization. Additionally, CEACAM1 has been implicated in infection with influenza virus H5N1 and with filial nematodes such as Wucheria bancrofti.

Human CEACAM1 (hCEACAM1) is a single pass type I transmembrane protein expressed as 12 alternatively spliced isoforms that all contain an N-terminal V set fold of the immunoglobulin superfamily (IgV) ectodomain followed by up to three type 2 constant immunoglobulin (IgC2) ectodomains (A1, B, A2), a transmembrane sequence, and a signaling cytoplasmic domain. Depending on splice variation, the cytoplasmic domain either includes a long (L) sequence inclusive of two immunoreceptor tyrosine-based inhibitory motifs (ITIMs) or a short (S) domain devoid of ITIMs that impart intracellular inhibitory or non-inhibitory signals, respectively.

CEACAM1 function is triggered by intercellular or trans binding of the IgV domain, resulting in higher order surface CEACAM1 oligomerization and subsequent intracellular signal transduction.

In contrast to other immunoreceptors such as the T cell inhibitory and mucin domain-containing protein 3 (TIM-3), programmed cell death protein 1 (PD-1), and programmed death-ligand 1 (PD-L1), CEACAM1 serves as its own primary ligand, owing to high affinity homophilic interactions of its unique IgV domain, as well as an important microbial receptor. At basal steady state, CEACAM1 alternates between monomeric and cis homodimeric forms on the cell surface.

The hCEACAM1 IgV domain contains 108 amino acids arranged in 9 beta strands (ABCC′C″DEFG) that fold into the conserved IgV anti-parallel beta-sandwich tertiary structure adopted by other IgV-containing immunoreceptors including TIM-3, PD-1, and PD-L1. The opposing ABED and GFCC′ faces of the CEACAM1 beta-sandwich are tethered by an internal salt bridge (R64:D82) that mimics a stabilizing covalent disulfide linkage found in most Ig domains. The more dominant oligomeric form is the high-affinity GFCC′-mediated homodimer that is conserved among other IgV domain-containing proteins. Unique to the hCEACAM1 IgV GFCC′ surface is the prominent protrusion of the CC′ loop that differs notably from the ordered β-hairpin observed in other described IgV structures. The displaced CC′ loop forms a cleft with the FG loop that exposes the key residues F29, S32, Y34, V39, G41, Q44, Q89, 191, N97, and E99 critical in mediating homophilic CEACAM1 interactions. The structure of hCEACAM1, including the structure of the GF-CC′ face, is presented in Huang et al., Nature 517: 386-390 (2015), which is incorporated herein by reference in its entirety.

The hCEACAM1 IgV GFCC′ face represents the primary face involved in homodimer and heterodimer formation. When hCEACAM1 transitions from a dimer to monomer, through disruption of CC′ loop interactions, there is higher thermal motion and dynamic conformations within the CC′ and FG loops that accommodate a monomeric hCEACAM1 species that is readily amenable to participating in both homophilic and heterophilic interactions. Homophilic interactions mediated by the GFCC′ face of a hCEACAM1 monomer with a neighboring hCEACAM1 monomer either in cis or trans return the destabilized hCEACAM1 monomers to a more thermally stable dimeric form. The GFCC′ face-stabilized homodimer participates in higher order oligomer formation, through minor interactions mediated though the ABED face. Selective targeting of the GFCC′ face of hCEACAM1 can disrupt the formation of hCEACAM1 homodimers and complexes with other ligands such as hTIM3, PD-1, and PD-L1, respectively, using therapeutic agents that exceed the natural homodimeric and heterodimeric affinities. Additional information on the GFCC′ face of CEACAM1 can be found in Huang, Yu-Hwa et al. 2015. CEACAM1 regulates TIM-3-mediated tolerance and exhaustion. Nature 517(7534): 386-390; Gandhi et al. 2021. Structural basis of the dynamic human CEACAM1 monomer-dimer equilibrium. Communications Biology 4, Article number: 360; and Gandhi, A. K. et al. 2022. Structural analysis of human CEACAM1 oligomerization. Communications Biology 5, Article number 1042; which are incorporated herein by reference in their entireties.

### CEACAM1-Related Diseases or Disorders

As used herein, a CEACAM1-related disease or disorder is one in which CEACAM1-mediated signaling contributes to disease pathology, and which can be effectively treated to reduce such pathology by inhibiting CEACAM1 function, including inhibiting interactions of CEACAM1 with one or more CEACAM1 ligands. Symptoms or markers of a CEACAM1-related disease or disorder include altered infiltration pattern of immune cells in the microenvironment, unresponsive T cells, low levels of T cells, an increase in regulatory T cells, and/or increased expression of CEACAM1.

Examples of a CEACAM1-related disease or disorder include, but are not limited to, a cancer or a chronic infection. In some embodiments the subject has cancer. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, as well as other carcinomas and sarcomas.

The response to anti-cancer therapy relates to any response of the subject's cancer or tumor(s) to therapy, preferably to a change in tumor mass and/or volume after initiation of therapy. Tumor response can be assessed, for example, where the size of a tumour after intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response can also be assessed by caliper measurement or pathological examination of the tumour after biopsy or surgical resection. Response can be recorded in a quantitative fashion like percentage change in tumour volume or cellularity or using a semi-quantitative scoring system such as residual cancer burden (Symmans et al., J. Clin. Oncol. (2007) 25:4414-5 4422) or Miller-Payne score (Ogston et al., Breast (Edinburgh, Scotland) (2003) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response can be performed early after the onset of therapy, e.g., after a few hours, days, weeks or preferably after a few months. A typical endpoint for response assessment is upon termination of therapy or upon surgical removal of residual tumor cells and/or the tumor bed.

Additional criteria for evaluating the response to anti-cancer therapies are related to survival, which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of such survival can be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

In some embodiments, the CEACAM1-binding antibodies are administered in combination with one or more additional anti-cancer/anti-tumor agents or medicaments. In this context, an “anti-tumor medicament” or, equivalently, a “cancer medicament”, refers to an agent which is administered to a subject for the purpose of treating a cancer. Treating cancer can include preventing the development or progression of a cancer, reducing the symptoms of cancer, and/or inhibiting the growth of an established cancer. Various types of medicaments for the treatment of cancer are described herein and/or known in the art. For the purpose of this specification, cancer medicaments can be classified as chemotherapeutic agents, immunotherapeutic agents, cancer vaccines, hormone therapies, and biological response modifiers.

Cancer medicaments function in a variety of ways. Some cancer medicaments work by targeting physiological mechanisms that are specific to tumor cells. Examples include the targeting of specific genes and their gene products (primarily proteins) which are mutated or over- or inappropriately expressed in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms which are altered in cancer cells. Targeting of cancer cells via the epitopes expressed on their cell surface is accomplished, for example, through the use of monoclonal antibodies.

Other cancer medicaments target cells other than cancer cells. For example, some medicaments prime the immune system to attack tumor cells (e.g., cancer vaccines and immune checkpoint inhibitors). While this section is relevant to therapies that can be combined with anti-CEACAM1 antibodies, it is noted that anti-CEACAM1 therapy promotes an anti-cancer immune response by interfering with the immunosuppressive function of CEACM1, and is therefore a type of immunotherapy. Still other medicaments, such as angiogenesis inhibitors, function by attacking the blood supply of solid tumors. Angiogenesis inhibitors include basic FGF (b-FGF), VEGF, angiopoietins, angiostatin, endostatin, TNF-α, TNP-470, thrombospondin-1, platelet factor 4, CAI, and certain members of the integrin family of proteins. Since the most malignant cancers are able to metastasize (i.e., exit the primary tumor site and seed a distal tissue, thereby forming a secondary tumor), medicaments that impede this metastasis are also useful in the treatment of cancer. One category of this type of medicament is a metalloproteinase inhibitor, which inhibits the enzymes used by the cancer cells to exist the primary tumor site and extravasate into another tissue.

As used herein, chemotherapeutic agents embrace all other forms of cancer medicaments which do not fall into the categories of immunotherapeutic agents or cancer vaccines. Chemotherapeutic agents as used herein encompass both chemical and biological agents. These agents function to inhibit a cellular activity upon which the cancer cell or tumor depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation.

Chemotherapeutic agents which are currently in development or in use in a clinical setting include, without limitation: 5-FU Enhancer, 9-AC, AG2037, AG3340, Aggrecanase Inhibitor, Aminoglutethimide, Amsacrine (m-AMSA), Angiogenesis Inhibitor, Anti-VEGF, Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorombucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p′-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL®), PARP Inhibitors, PD 183805, Pentostatin (2′ deoxycoformycin), PKC 412, Plicamycin, Procarbazine HCl, PSC 833, Ralitrexed, RAS Famesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/Uracil), Valrubicin, VEGF/b-FGF Inhibitors, Vinblastine sulfate, Vindesine sulfate, VX-710, VX-853, YM 116, ZD 0101, ZD 0473/Anormed, ZD 1839, and ZD 9331, among others.

In some preferred embodiments the subject has an infection or is at increased risk of having an infection. An infection is a disease or condition attributable to the presence in a host of a foreign organism or agent that reproduces within the host. Infections typically involve breach of a normal mucosal or other tissue barrier by an infectious organism or agent. A subject that has an infection is a subject having objectively measurable infectious organisms or agents present in the subject's body. A subject at increased risk of having an infection is a subject that is predisposed to develop an infection. Such a subject can include, for example, a subject with a known or suspected exposure to an infectious organism or agent. A subject at increased risk of having an infection also can include a subject with a condition associated with impaired ability to mount an immune response to an infectious organism or agent, e.g., a subject with a congenital or acquired immunodeficiency, a subject undergoing radiation therapy or chemotherapy, a subject with a burn injury, a subject with a traumatic injury, a subject undergoing surgery or other invasive medical or dental procedure.

Infections are broadly classified as bacterial, viral, fungal, or parasitic based on the category of infectious organism or agent involved. Other less common types of infection are also known in the art, including, e.g., infections involving rickettsiae, mycoplasmas, and agents causing scrapie, bovine spongiform encephalitis (BSE), and prion diseases (e.g., kuru and Creutzfeldt-Jacob disease). Examples of bacteria, viruses, fungi, and parasites which cause infection are well known in the art. An infection can be acute, subacute, chronic, or latent, and it can be localized or systemic. Furthermore, an infection can be predominantly intracellular or extracellular during at least one phase of the infectious organism's or agent's life cycle in the host.

Bacteria include both Gram negative and Gram positive bacteria. Examples of Gram positive bacteria include, but are not limited to Pasteurella species, Staphylococci species, and Streptococcus species. Examples of Gram negative bacteria include, but are not limited to, Escherichia coli, Pseudomonas species, and Salmonella species. Specific examples of infectious bacteria include but are not limited to: Helicobacter pylori, Borrelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (Anaerobic spp.), Streptococcus pneumoniae, pathogenic Campylobacter spp., Enterococcus spp., Haemophilus influenzae, Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that have been found to cause infections in humans include but are not limited to: Retroviridae (e.g., human immunodeficiency viruses, such as HIV-1 (also referred to as HTLV-III), HIV-2, LAV or HTLV-III/LAV, or HIV-III, and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=enterally transmitted; class 2=parenterally transmitted (i.e., Hepatitis C); Norwalk and related viruses, and astroviruses).

Examples of fungi include: Aspergillus spp., Blastomyces dermatitidis, Candida albicans, other Candida spp., Coccidioides immitis, Cryptococcus neoformans, Histoplasma capsulatum, Chlamydia trachomatis, Nocardia spp., Pneumocystis carinii.

Parasites include but are not limited to blood-borne and/or tissue parasites such as Anaplasma phagocytophilum, Babesia microti, Babesia divergens, Entamoeba histolytica, Giardia lamblia, Leishmania tropica, Leishmania spp., Leishmania braziliensis, Leishmania donovani, Plasmodium falciparum, Plasmodium malariae, Plasmodium ovale, Plasmodium vivax, and Toxoplasma gondii, Trypanosoma gambiense and Trypanosoma rhodesiense (African sleeping sickness), Trypanosoma cruzi (Chagas' disease), and Toxoplasma gondii, flat worms, round worms.

In some embodiments, methods described herein further involve administering to the subject an antibacterial medicament. An antibacterial medicament is an agent that kills or inhibits the growth or function of bacteria. Antibacterial agents which are effective for killing or inhibiting a wide range of bacteria are often referred to as broad spectrum antibiotics. Other types of antibacterial agents are predominantly effective against Gram-positive bacteria or Gram-negative bacteria. These types of antibacterial agents are frequently referred to as narrow spectrum antibiotics. Other antibacterial agents which are effective against a single organism or disease and not against other types of bacteria, are often referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and/or competitive inhibitors.

Antibacterial agents useful in the methods described herein include but are not limited to natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, 19mmune19a119, cefoxitin, cefotaxime, cefsulodin, cefetamet, cefixime, ceftriaxone, cefoperazone, 19mmune19a119r, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and Zorbamycin.

In some embodiments the methods further involve administering to the subject an antiviral medicament. An antiviral medicament is a compound which prevents infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g., amantadine), synthesis or translation of viral mRNA (e.g., interferon), replication of viral RNA or DNA (e.g., nucleoside analogues), maturation of new virus proteins (e.g., protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), ganciclovir (useful for the treatment of cytomegalovirus), idoxuridine, ribavirin (useful for the treatment of respiratory syncytial virus infection), dideoxyinosine, dideoxycytidine, and zidovudine (azidothymidine).

The interferons are cytokines which are secreted by virus-infected cells as well as immune cells. The interferons function by binding to specific receptors on cells adjacent to the infected cells, causing the change in the cell which protects it from infection by the virus. IFN-α and IFN-β also induce the expression of Class I and Class II MHC molecules on the surface of infected cells, resulting in increased antigen presentation for host immune cell recognition. These interferons are available as recombinant forms and have been used for the treatment of chronic hepatitis B and C infection. At the dosages which are effective for antiviral therapy, interferons sometimes have severe side effects such as fever, malaise and weight loss.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immune globulin therapy and hyper-immune globulin therapy. Normal immune globulin therapy utilizes an antibody product which is prepared from the serum of normal blood donors and pooled. This pooled product contains low titers of antibody to a wide range of human viruses, such as hepatitis A, parvovirus and enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immuno-compromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncytial virus infections).

Thus, antiviral medicaments useful in combination with anti-CEACAM1 antibody agents as described herein include but are not limited to immunoglobulins, amantadine, interferon, nucleoside analogues, and protease inhibitors. Specific examples of antiviral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Molnupiravir; Nevirapine; Nirmatrelvir; Penciclovir; Pirodavir; Ribavirin; Rimantadine Hydrochloride; Ritonavir; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In some embodiments the methods described herein further involve administering to the subject an antifungal medicament. An antifungal medicament is an agent that kills or inhibits the growth or function of infective fungi. Anti-fungal medicaments are sometimes classified by their mechanism of action. Some anti-fungal agents function as cell wall inhibitors by inhibiting glucose synthase. These include, but are not limited to, basiungin/ECB. Other antifungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other antifungal agents function by breaking down chitin (e.g., chitinase) or immunosuppression (501 cream).

Thus, the antifungal medicaments useful in the methods described herein include but are not limited to imidazoles, 501 cream, and Acrisorcin, Ambruticin, Amorolfine, Amphotericin B, Azaconazole, Azaserine, Basifungin, BAY 38-9502, Bifonazole, Biphenamine Hydrochloride, Bispyrithione Magsulfex, Butenafine, Butoconazole Nitrate, Calcium Undecylenate, Candicidin, Carbol-Fuchsin, Chitinase, Chlordantoin, Ciclopirox, Ciclopirox Olamine, Cilofungin, Cisconazole, Clotrimazole, Cuprimyxin, Denofungin, Dipyrithione, Doconazole, Econazole, Econazole Nitrate, Enilconazole, Ethonam Nitrate, Fenticonazole Nitrate, Filipin, FK 463, Fluconazole, Flucytosine, Fungimycin, Griseofulvin, Hamycin, Isoconazole, Itraconazole, Kalafungin, Ketoconazole, Lomofungin, Lydimycin, Mepartricin, Miconazole, Miconazole Nitrate, MK 991, Monensin, Monensin Sodium, Naftifine Hydrochloride, Neomycin Undecylenate, Nifuratel, Nifurmerone, Nitralamine Hydrochloride, Nystatin, Octanoic Acid, Orconazole Nitrate, Oxiconazole Nitrate, Oxifungin Hydrochloride, Parconazole Hydrochloride, Partricin, Potassium Iodide, Pradimicin, Proclonol, Pyrithione Zinc, Pyrrolnitrin, Rutamycin, Sanguinarium Chloride, Saperconazole, Scopafungin, Selenium Sulfide, Sertaconazole, Sinefungin, Sulconazole Nitrate, Terbinafine, Terconazole, Thiram, Ticlatone, Tioconazole, Tolciclate, Tolindate, Tolnaftate, Triacetin, Triafungin, UK 292, Undecylenic Acid, Viridofulvin, Voriconazole, Zinc Undecylenate, and Zinoconazole Hydrochloride.

In some embodiments the methods described herein further involve administering to the subject an antiparasitic medicament. An antiparasitic medicament is an agent that kills or inhibits the growth or function of infective parasites. Examples of antiparasitic medicaments, also referred to as parasiticides, useful in the methods described herein include but are not limited to albendazole, amphotericin B, benznidazole, bithionol, chloroquine HCl, chloroquine phosphate, clindamycin, dehydroemetine, diethylcarbamazine, diloxanide furoate, doxycycline, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, thiabendazole, tinidazole, trimethroprim-sulfamethoxazole, and tryparsamide, some of which are used alone or in combination with others.

Conditions that might benefit from anti-hCEACAM1 monoclonal antibody therapy include those requiring enhancement of B and T cells responses to immunogens and include cancers, for example melanoma, pancreatic, thyroid, lung, colorectal, squamous, prostate, breast, bladder and gastric cancers.

### Antibodies

Antibodies that specifically bind CEACAM1 can be used to inhibit CEWACAM1 activity and thereby treat diseases or disorders in which CEACAM1 is involved in their pathologies. The term “antibody” is used in the broadest sense and includes monoclonal antibodies (including full length or intact monoclonal antibodies), polyclonal antibodies, multivalent antibodies, multispecific antibodies (e.g., bispecific antibodies), antibody fragments, and antigen-binding portions thereof (e.g., paratopes, CDRs), so long as they exhibit the desired biological activity and specificity.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). VH refers to the variable domain of the heavy chain. VL refers to the variable domain of the light chain. The amino acid positions assigned to CDRs and FRs may be defined according to the numbering conventions of Kabat or Chothia. The term “framework regions” (FR) refers to those variable domain residues other than the CDR residues. (Kabat et al., Sequences of Proteins of Immunological Interest, 5^{th} Ed. Public Health Service, National Institutes of Health, Bethesda, Md. (1987, 1991)) or Chothia (Chothia & Lesk 196 J. Mol. Biol. 901 (1987)).

The “Complementarity Determining Regions” (CDRs) of an antibody are the portions of the antibody variable domain that are (typically) involved in antigen binding. Each variable domain typically has three CDR regions identified as CDR1, CDR2 and CDR3. Each CDR can comprise amino acid residues from a CDR as defined by e.g. Kabat (i.e., about residues 24-34 (L1), 50-56 (L2) and 89-97 (L3) in the light chain variable domain and 31-35 (H1), 50-65 (H2) and 95-102 (H3) in the heavy chain variable domain. Each CDR can also comprise amino acid residues from a “hypervariable loop” (i.e., about residues 26-32 (LI), 50-52 (L2) and 91-96 (L3) in the light chain variable domain and 26-32 (H1), 53-55 (H2) and 96-101 (H3) in the heavy chain variable domain (Chothia). In some instances, a CDR can include amino acids from both a CDR region defined according to Kabat and a hypervariable loop. The Kabat residue designations do not always correspond directly with the linear numbering of the amino acid residues (primary amino acid sequence). The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues can be determined for a given antibody or antigen-binding fragment thereof by alignment of residues of homology in the sequence of the antibody or antigen-binding fragment thereof with a “standard” Kabat numbered sequence. An example of how the Kabat numbering relates to the primary amino acid sequence of an antibody can be seen in PCT/US2019/065212, which is incorporated by reference in its entirety. Alternatively, a CDR can be defined according to the ImMunoGeneTics (IMGT) system (Lefranc, M.-P. et al., Dev. Comp. Immunol., 27, 55-77 (2003)).

In one embodiment, the antibody or antigen-binding fragment thereof, which binds to the GF-CC′ face of the CEACAM1 protein comprises a heavy chain variable region and a light chain variable region, wherein each of the heavy chain and the light chain variable regions comprises a CDR1, CDR2, and CDR3, and the sequence of CDR1 of the heavy chain variable region (CDR1H) comprises the sequence X₁HX₂X₃S (SEQ ID NO: 1); X₁ is A, D, N, or S; X₂ is A or G; and X₃ is an amino acid with a hydrophobic side chain including I or M; the sequence of CDR2 of the heavy chain variable region (CDR2H) comprises the sequence TISSGGTYTYYPDSVKG (SEQ ID NO:2); the sequence of CDR3 of the heavy chain variable region (CDR3H) comprises the sequence HX₄X₅DYX₆PX₇WFAX₈ (SEQ ID NO:3); X₄ is D, G, or P; X₅ is F or P; X₆ is D or F; X₇ is A or Y; and X₈ is L, H, or F; the sequence of CDR1 of the light chain variable region (CDR1L) comprises the sequence RANSAVSYMY (SEQ ID NO:4); the sequence of CDR2 of the light chain variable region (CDR2L) comprises the sequence LTSNRAT (SEQ ID NO:5); and the sequence of CDR3 of the light chain variable region (CDR3L) comprises the sequence QQX₉X₁₀X₁₁X₁₂PX₁₃T (SEQ ID NO:6); X₉ is W or N; X₁₀ is S or T; X₁₁ is A or an amino acid with a neutral hydrophilic side chain including S, N, and T; X₁₂ is L, F, or N; and X₁₃ is P or F.

In one embodiment, the region comprises SEQ ID NO:2; the sequence of CDR3 of the heavy chain variable region comprises SEQ ID NO:8; the sequence of CDR1 of the light chain variable region comprises SEQ ID NO:4; the sequence of CDR2 of the light chain variable region comprises SEQ ID NO:5; and the sequence of CDR3 of the light chain variable region comprises SEQ ID NO:10.

In one embodiment, the CEACAM1 antibody or antigen-binding fragment thereof comprises six CDRs, wherein: the sequence of CDR1 of the heavy chain variable region comprises SEQ ID NO:7; the sequence of CDR2 of the heavy chain variable region comprises SEQ ID NO:2; the sequence of CDR3 of the heavy chain variable region comprises SEQ ID NO:8; the sequence of CDR1 of the light chain variable region comprises SEQ ID NO:14; the sequence of CDR2 of the light chain variable region comprises SEQ ID NO:5; and the sequence of CDR3 of the light chain variable region comprises SEQ ID NO:9.

According to certain embodiments, the antibodies and antigen-binding fragments thereof also feature humanized frameworks for reduced immunogenicity. In certain embodiments, the CDRs of the antibody or antigen-binding fragment thereof are located in frameworks obtained from a human antibody or antigen-binding fragment thereof. In other embodiments, surface-exposed framework residues of the contemplated antibody or antigen-binding fragment thereof are replaced with framework residues of a human antibody or antigen-binding fragment thereof. The CDRs can also be located in murine or humanized frameworks linked to human constant regions (i.e., chimeric antibodies). In a preferred embodiment, the CDRs of an antibody or antigen-binding fragment thereof are located in frameworks that are a composite of two or more human antibodies. In such embodiments, the antibodies or antigen-binding fragments thereof comprise two or more sequence segments (“composites”) derived from V-regions of unrelated human antibodies that are selected to maintain monoclonal antibody sequences important for antigen binding of the starting precursor anti-human CEACAM1 monoclonal antibody, and which have all been filtered for the presence of potential T cell epitopes, e.g., using “in silico tools” (Holgate & Baker, IDrugs. 2009 April; 12(4):233-7). The close fit of human sequence segments with all sections of the starting antibody V regions and the elimination of CD4+ T cell epitopes prior to synthesis of the antibody or antigen-binding fragment thereof allow this technology to limit or circumvent immunogenicity while maintaining optimal affinity and specificity through the prior analysis of sequences necessary for antigen-specificity (Holgate & Baker, 2009).

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree of identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at ncbi.nlm.nih.gov/blast/). In comparing sequences, these methods account for various substitutions, deletions, and other modifications.

In another aspect, the CEACAM1 binding antibody or antigen-binding fragment thereof that binds to the GF-CC′ face thereof comprises
(i) a heavy chain variable domain comprising a sequence that is at least 90%, at least 95%, at least 96%, at least 97%, at least 98%, or at least 99% identical to a heavy chain variable domain sequence of SEQ ID NO: 11; and/or
(ii) a light chain variable domain comprising a sequence that is at least 90%, at least 95%, at least 96%, at least 97%, at least 98%, or at least 99% identical to the light chain variable domain sequence of SEQ ID NO:12.

In some embodiments, amino acid sequence modification(s) of the antibodies or antigen-binding fragments thereof described herein that bind to CEACAM1 are contemplated. Amino acid sequence variants of the antibody or antigen-binding fragment thereof are prepared by introducing appropriate nucleotide changes into the nucleic acid encoding the antibody or antigen-binding fragment thereof, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, provided that the final construct possesses the desired characteristics, e.g., binding specificity, inhibition of biological activity.

One type of variant is a conservative amino acid substitution variant. These variants have at least one amino acid residue in the antibody or antigen-binding fragment thereof replaced by a different residue that has similar side chain properties. Amino acids can be grouped according to similarities in the properties of their side chains (see Lehninger, BIOCHEMISTRY (2nd ed., Worth Publishers, New York, 1975):
(1) non-polar: Ala (A), Val (V), Leu (L), Ile (I), Pro (P), Phe (F), Trp (W), Met (M);
(2) uncharged polar: Gly (G), Ser (S), Thr (T), Cys (C), Tyr (Y), Asn (N), Gln (Q);
(3) acidic: Asp (D), Glu (E);
(4) basic: Lys (K), Arg (R), His (H).
   As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser (S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).
   As such, a non-limiting example for a conservative amino acid substitution is one that replaces a hydrophobic amino acid with another hydrophobic amino acid.

Further contemplated are amino acid sequence insertions, which can include amino- and/or carboxyl-terminal fusions ranging in length from one residue to polypeptides containing a hundred or more residues, as well as intrasequence insertions of single or multiple amino acid residues. Examples of terminal insertions include an antibody or antigen-binding fragment thereof with an N-terminal methionyl residue or the antibody or antigen-binding fragment thereof fused to a cytotoxic polypeptide. Other insertional variants of the antibody or antigen-binding fragment thereof include the fusion to the N- or C-terminus of the antibody or antigen-binding fragment thereof to an enzyme or a polypeptide which increases the serum half-life of the antibody or antigen-binding fragment thereof, such as, for example, biotin.

Any cysteine residue not involved in maintaining the proper conformation of the antibodies or antigen-binding fragments thereof that bind to CEACAM1 also can be substituted, for example with a serine or an alanine, to improve the oxidative stability of the molecule and prevent aberrant crosslinking.

Conversely, cysteine bond(s) can be added to the antibody or antigen-binding fragment thereof to improve its stability (particularly where the antibody or antigen-binding fragment thereof is an antibody fragment such as an Fv fragment).

In some embodiments, the antibodies or antigen-binding fragments thereof have amino acid alterations that alter the original glycosylation pattern of the antibody or antigen-binding fragment thereof. By “altering the original glycosylation pattern” is meant deleting one or more carbohydrate moieties found in the antibody or antigen-binding fragment thereof, and/or adding one or more glycosylation sites that are not present in the antibody or antigen-binding fragment thereof. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. Addition of glycosylation sites to the antibodies or antigen-binding fragments thereof that bind to CEACAM1 is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody or antigen-binding fragment thereof (for O-linked glycosylation sites).

In some embodiments, the CEACAM1 antibodies or antigen-binding fragments thereof provided herein are deglycosylated or aglycosylated. In some embodiments, the CEACAM1 antibody or antigen-binding fragment thereof lacks a glycosylation site in the CDR1 of the variable light chain. In some embodiments, the CEACAM1 antibody or antigen-binding fragment thereof lacks an N-X-S/T consensus sequence in the CDR1 of the variable light chain. In some embodiments, the CEACAM1 antibody or antigen-binding fragment thereof has a mutation in CDR residues 26 and/or 29 (Kabat numbering) of the CDR1 of the variable light chain. Where the antibody or antigen-binding fragment thereof comprises an Fc region, the carbohydrate(s) attached thereto can be altered. For example, antibodies with a mature carbohydrate structure that lacks fucose attached to an Fc region of the antibody or antigen-binding fragment thereof are described. See, e.g., U.S. Patent Pubs. No. 2003/0157108; No. 2004/0093621. Antibodies with a bisecting N-acetylglucosamine (G1cNAc) in the carbohydrate attached to an Fc region of the antibody or antigen-binding fragment thereof are referenced in WO 03/011878; U.S. Pat. No. 6,602,684. Antibodies with at least one galactose residue in the oligosaccharide attached to an Fc region of the antibody or antigen-binding fragment thereof are reported in WO 97/30087. See also WO 98/58964; WO 99/22764 concerning antibodies with altered carbohydrate attached to the Fc region thereof.

In some embodiments, it can be desirable to modify the antibodies or antigen-binding fragment thereof that bind to CEACAM1 described herein with respect to effector function, e.g., so as to enhance antigen-dependent cell-mediated cyotoxicity (ADCC) and/or complement dependent cytotoxicity (CDC) of the antibody or antigen-binding fragment thereof. This can be achieved by introducing one or more amino acid substitutions in an Fc region of the antibody or antigen-binding fragment thereof. Alternatively or additionally, one or more cysteine residues can be introduced in the Fc region, thereby allowing interchain disulfide bond formation in this region. The homodimeric antibody or antigen-binding fragment thereof thus generated can have improved internalization capability and/or increased complement-mediated cell killing and antibody-dependent cellular cytotoxicity (ADCC). See Caron et al., 176 J. Exp. Med. 1191 (1992); Shopes, 148 J. Immunol. 2918 (1992). Homodimeric antibodies with enhanced anti-tumor activity can also be prepared using heterobifunctional cross-linkers as described in Wolff et al., 53 Cancer Res. 2560 (1993). Alternatively, an antibody or antigen-binding fragment thereof can be engineered which has dual Fc regions and can thereby have enhanced complement lysis and ADCC capabilities. See Stevenson et al., 3 Anti-Cancer Drug Design 219 (1989).

For example, WO 00/42072 describes antibodies with improved ADCC function in the presence of human effector cells, where the antibodies comprise amino acid substitutions in the Fc region thereof. Preferably, the antibody or antigen-binding fragment thereof with improved ADCC comprises substitutions at positions 298, 333, and/or 334 of the Fc region (Eu numbering of residues). Typically, the altered Fc region is a human IgG1 Fc region comprising or consisting of substitutions at one, two or three of these positions. Such substitutions are optionally combined with substitution(s) which increase Clq binding and/or CDC. Substitutions include an Asn297Ala mutation in IgG1 Fc.

Antibodies with altered Clq binding and/or complement dependent cytotoxicity (CDC) are described in WO 99/51642, U.S. Pat. Nos. 6,194,551, 6,242,195, 6,528,624, and 6,538,124. The antibodies comprise an amino acid substitution at one or more of amino acid positions 270, 322, 326, 327, 329, 313, 333 and/or 334 of the Fc region thereof (Eu numbering of residues).

Antibodies with improved binding to the neonatal Fc receptor (FcRn), and increased half-lives, are described in WO 00/42072 and U.S. Patent Pub. No. 2005/0014934. These antibodies comprise an Fc region with one or more substitutions therein which improve binding of the Fc region to CEACAM1. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising antibody variant with improved CEACAM1 binding comprises amino acid substitutions at one, two or three of positions 307, 380 and 434 of the Fc region thereof (Eu numbering of residues). In one embodiment, the antibody or antigen-binding fragment thereof has 307/434 mutations. Engineered antibodies that bind to CEACAM1 with three or more (e.g., four) functional antigen binding sites are also contemplated. See, e.g., U.S. Patent Pub. No. US 2002/0004587.

### Antibody Fragments and Types

In some embodiments of the aspects described herein, the CEACAM1 antibody fragment is a Fab fragment, which comprises or consist essentially a variable (VL) and constant (CL) domain of the light chain and a variable domain (VH) and the first constant domain (CH1) of the heavy chain.

In some embodiments of the aspects described herein, the CEACAM1 antibody fragment is a Fab′ fragment, which refers to a Fab fragment having one or more cysteine residues at the C-terminus of the CH1 domain.

In some embodiments of the aspects described herein, the CEACAM1 antibody fragment is an Fd fragment comprising or consisting essentially of VH and CH1 domains.

In some embodiments of the aspects described herein, the CEACAM1 antibody portion is an Fd′ fragment comprising VH and CH1 domains and one or more cysteine residues at the C-terminus of the CH1 domain.

Single-chain Fv or scFv antibody fragments comprise or consist essentially of the VH and VL domains of antibody, such that these domains are present in a single polypeptide chain. Generally, an Fv polypeptide further comprises a polypeptide linker between the VH and VL domains, which allows the scFv to form the desired structure for antigen binding. See, for example, Pluckthun, 113 Pharmacology Monoclonal Antibodies 269 (Rosenburg & Moore, eds., Springer-Verlag, New York, 1994). Accordingly, in some embodiments of the aspects described herein, the CEACAM1 antibody fragment is a Fv fragment comprising or consisting essentially of the VL and VH domains of a single arm of an antibody.

In some embodiments of the aspects described herein, the CEACAM1 antibody portion is a diabody comprising two antigen binding sites, comprising a heavy chain variable domain (VH) connected to a light chain variable domain (VL) in the same polypeptide chain.

In some embodiments of the aspects described herein, the CEACAM1 antibody portion is a dAb fragment comprising or consisting essentially of a VH domain.

In some embodiments of the aspects described herein, the CEACAM1 antibody portion is a F(ab′)2 fragment, which comprises a bivalent fragment comprising two Fab′ fragments linked by a disulfide bridge at the hinge region.

Linear antibodies are described in Zapata et al., Protein Engin., 8(10):1057-1062 (1995). Briefly, these antibodies comprise a pair of tandem Fd segments (VH-CH1-VH-CH1), which, together with complementary light chain polypeptides, form a pair of antigen binding regions. Linear antibodies can be bispecific or monospecific. In some embodiments of the aspects described herein, the CEACAM1 antibody fragment is a linear antibody comprising a pair of tandem Fd segments (VH-CH1-VH-CH1) which, together with complementary light chain polypeptides, form a pair of antigen binding regions.

Various techniques have been developed and are available for the production of antibody fragments. Traditionally, these fragments were derived via proteolytic digestion of intact antibodies. See, e.g., Morimoto et al., 24 J. Biochem. Biophys. Meths. 107 (1992); Brennan et al., 229 Science 81 (1985). However, these fragments can now be produced directly by recombinant host cells. For example, antibody fragments can be isolated from the antibody phage libraries discussed herein. Alternatively, Fab′-SH fragments can be directly recovered from E. coli and chemically coupled to form F(ab′)2 fragments (Carter et al., 1992). According to another approach, F(ab′)2 fragments can be isolated directly from recombinant host cell culture. Other techniques for the production of antibody fragments will be apparent to the skilled practitioner. In other embodiments, the antibody fragment of choice is a single chain Fv fragment (scFv). See, for example, WO 93/16185.

In one embodiment, the antibody is a bispecific antibody comprising a complementary region that binds to CEACAM1 and a complementary region that binds to PD-1.

In one embodiment, the antibody is a bispecific antibody comprising a complementary region that binds to CEACAM1 and a complementary region that binds to PD-L1.

Contemplated antibodies or antigen-binding fragments may have all types of constant regions, including IgM, IgG, IgD, and IgE, and any isotype, including IgG1, IgG2, IgG3, and IgG4. In one embodiment, the human isotype IgG1 is used. In another embodiment, the human isotype IgG4 is used. Light chain constant regions can be a, or K. The antibody or antigen-binding fragment thereof may comprise sequences from more than one class or isotype.

### Antibody Binding

The methods described herein permit the determination of whether a given subject's CEACAM1 (or whether a subject's cancer's CEACAM1) will be inhibited by antibodies that bind the GF-CC′ face of CEACAM1. Where the antibodies bind efficiently to wild-type but either do not bind or bind with reduced affinity to certain allelic variants, one can predict based on CEACAM1 genotype whether a given subject's CEACAM1-mediated disease or disorder would be likely to respond (or not) to the antibodies, or would potentially require higher dosages or more frequent administration of the antibodies. The human CEACAM1 gene produces 11 isoforms by alternative splicing. Each isoform has one variable (V)-like Ig domain at the amino (N) end of the protein. With the exception of CEACAM1-1L and CEACAM1-1S isoforms, the various isoforms also have 2 or 3 constant C2-like Ig domains. Eight CEACAM1 isoforms are anchored to the cellular membrane via a transmembrane domain and three CEACAM1 isoforms (CEACAM1-4C1, -3 and -3C2) lack the transmembrane domain and are secreted. Two isoforms (CEACAM1-3AL and -3AS) have an Alu family repeat sequence (A) between the constant C2-like Ig domains and the transmembrane domain. The transmembrane CEACAM1 isoforms also possess a long (L) or short (S) cytoplasmic domain determined by inclusion or exclusion of CEACAM1 exon 7 in the message. The CEACAM1 L cytoplasmic domain has two ITIM motifs, which are unique to CEACAM1 among the CEACAM family members. In one aspect, therapeutic anti-CEACAM1 antibodies or antigen-binding fragments thereof, including the antibodies described herein by their structural characteristics, bind to the GF-CC′ face of the extracellular, variable (V)-like Ig domain at the amino (N) end of the protein (N-domain) of CEACAM1, a domain that is common to all isoforms of CEACAM1, including CEACAM1 isoforms 1L, 1S, 3L, 3S, 4L, 4S, 3A1, 3AS, 3, 4C1, and 4C2. In some embodiments, the provided antibodies and antigen-binding fragments thereof bind to human CEACAM1. In some embodiments, the provided antibodies and antigen-binding fragments thereof bind to mammalian CEACAM1. The sequence of the full-length form of CEACAM1 (NCB1 Reference Sequence NP_p01703.2; UNIPROT ID P13688) is provided as SEQ ID NO: 15 (signal sequence: residues 1-34 of SEQ ID NO: 15; Ig-V N domain: residues 35-142 of SEQ ID NO:15. The mature form of CEACAM1 (without signal sequence) is provided as SEQ ID NO:13.

As used herein, “binding” of an antibody or antigen binding fragment thereof to CEACAM1, an epitope on CEACAM1, or, in certain embodiments described below, particular residues on CEACAM1, includes the selective interaction of the antibody or antigen binding fragment thereof with CEACAM1. Binding therefore includes, e.g., primary and secondary interactions including hydrogen bonds, ionic interactions and salt bridges, as well as hydrophilic and hydrophobic interactions.

In certain embodiments, the CEACAM1 antibodies or antigen-binding fragments thereof described herein bind to CEACAM1 with a K_{D} of 10⁻⁵ to 10⁻¹² mol/l, 10⁻⁶ to 10⁻¹² mol/l, 10 to 10⁻¹² mol/l, 10⁻⁸ to 10⁻¹² mol/l, 10⁻⁹ to 10⁻¹² mol/l, 10⁻¹⁰ to 10⁻¹² mol/l, or 10⁻¹ to 10⁻¹² mol/l. In other embodiments, the CEACAM1 antibodies or antigen-binding fragments thereof described herein bind to CEACAM1 with a K_{D} of 10⁻⁵ to 10⁻¹¹ mol/l, 10⁻⁶ to 10⁻¹¹ mol/l, 10⁻⁷ to 10⁻¹¹ mol/l, 10⁻⁸ to 10⁻¹¹ mol/l, 10⁻⁹ to 10⁻¹¹ mol/l, or 10⁻¹⁰ to 10⁻¹¹ mol/l. In other embodiments, the CEACAM1 antibodies or antigen-binding fragments thereof described herein bind to CEACAM1 with a K_{D} of 10⁻⁵ to 10⁻¹⁰ mol/l, 10⁻⁶ to 10⁻¹⁰ mol/l, 10⁻⁷ to 10⁻¹⁰ mol/l, 10⁻⁸ to 10⁻¹⁰ mol/l, or 10⁻⁹ to 10⁻¹⁰ mol/l. In other embodiments, the CEACAM1 antibodies or antigen-binding fragments thereof described herein bind to CEACAM1 with a K_{D} of 10⁻⁵ to 10⁻⁸ mol/l, 10⁻⁶ to 10⁻⁸ mol/l, or 10⁻⁷ to 10⁻⁸ mol/l.

The “specificity” of an antibody refers to the ability of an antibody or antigen-binding fragment thereof, such as an anti-CEACAM1 antibody or antigen-binding fragment thereof, to recognize a given epitope within CEACAM1, while only having little or no detectable reactivity with non-CEACAM1 proteins or other portions of CEACAM1. Specificity can be relatively determined by competition assays or by epitope identification/characterization techniques described herein or their equivalents known in the art.

An epitope can be formed both from contiguous amino acids, or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a particular spatial conformation. An “epitope” includes the unit of structure conventionally bound by an immunoglobulin VH/VL pair. Epitopes define the minimum binding site for an antibody or antigen-binding fragment thereof, and thus represent the target of specificity of an antibody or antigen-binding fragment thereof. In the case of a single domain antibody, an epitope represents the unit of structure bound by a variable domain in isolation.

In a particular embodiment, the contemplated antibody or antigen-binding fragment specifically binds to the same epitope as antibody CP08H03/Vk8 S29A. In another embodiment, the contemplated antibody or antigen-binding fragment binds to the same epitope as CP08H03/CP08F05.

Additional description on the creation of the CP08 antibodies and the binding of the epitopes of antibodies CP08/Vk8 S29A and CP08H03/CP08F05 can be found in International Application No. PCT/US2019/065212, which is incorporated by reference herein in its entirety.

In one aspect, antibodies and antigen-binding fragments thereof, including the antibodies described herein by their structural characteristics, specifically bind to at least part of the homophilic binding domain on CEACAM1 (i.e. the portion of the CEACAM1 protein that is involved in formation of a CEACAM1:CEACAM1 homodimer), thereby blocking CEACAM1 homophilic interactions. In certain embodiments, the provided antibody or antigen-binding fragment thereof specifically binds to one or more of CEACAM1 residues that are contained in the CC′ and FG loops of CEACAM1 and that include a YQQN pocket at the CEACAM1:CEACAM1 dimer interface (i.e., Y34, Q44, Q89, N97 of SEQ ID NO:13), see Huang et al., Nature. 2015 Jan. 15; 517(7534):386-90.

As used herein, a “blocking” antibody or an “antagonist” antibody is one that inhibits or reduces biological activity of the antigen to which it binds. For example, in some embodiments, a CEACAM1 antagonist antibody or antigen-binding fragment thereof binds CEACAM1 and inhibits activity of CEACAM1 and/or binding of CEACAM1 to heterologous binding partners such as other CEACAM proteins or TIM-3. Inhibition of activity and inhibition of binding includes partial inhibition. Methods for the identification of CEACAM1 antibodies that block CEACAM1 homophilic and heterophilic interactions are described herein and are known to the those of skill in the art. For instance, competing, cross-blocking, and cross-blocked antibodies can be identified using any suitable method known in the art, including competition ELISAs or BIACORE® assays where binding of the competing or cross-blocking antibody to human CEACAM1 prevents the binding of an antibody disclosed herein or vice versa.

In certain embodiments, not all CDRs are directly involved in binding to the antigen. In one embodiment, four out of six CDRs of the CEACAM1 antibody or antigen-binding fragment thereof make contact with the antigen. In one embodiment, five out of six CDRs of the CEACAM1 antibody or antigen-binding fragment thereof make contact with the antigen. In one embodiment, six out of six CDRs of the CEACAM1 antibody or antigen-binding fragment thereof make contact with the antigen. In one embodiment, CDR2H, CDR3H, CDR1L, CDR2L, and CDR3L of the CEACAM1 antibody or antigen-binding fragment thereof are directly involved in binding to the antigen.

Despite the similarity of N-domains among CEACAM family members, in some embodiments, antibodies or antigen-binding fragments thereof provided herein, including the antibodies described herein by their structural characteristics, are selective for CEACAM1. By selectively targeting CEACAM1, embodiments of the invention may avoid undesired interfering e.g. with the broad activating function of CEACAM3.

The terms “selective” and “selectivity” herein refer to the preferential binding of an antibody or antigen-binding fragment thereof (i.e., a CEACAM1 antibody or antigen-binding fragment thereof), for a particular region, target, or peptide; typically a region or epitope in CEACAM1, as opposed to one or more other biological molecules, including other CEACAM family members.

In some embodiments, the CEACAM1 antibody or antigen-binding fragment thereof does not exhibit significant binding to CEACAM3, CEACAM5, CEACAM6 and/or CEACAM8. In some embodiments, the CEACAM1 antibody or antigen-binding fragment thereof does not exhibit detectable binding to CEACAM3, CEACAM5, CEACAM6 and/or CEACAM8. In some embodiments, the contemplated CEACAM1 antibody or antigen-binding fragment thereof binds CEACAM1 with an affinity that is at least 10 times, such as at least 100 times, and at least 1000 times, and up to 10,000 times or more stronger than the affinity with which the contemplated CEACAM1 antibody or antigen-binding fragment thereof binds to another target or polypeptide.

In some instances, the CEACAM1 antibody or antigen-binding fragment will not bind to CEACAM1. Lack of binding with CEACAM1 occurs when both alleles of the CEACAM1 gene are mutated or variant for A49, Q89, Q44, and/or Y34. These variations can be point mutations that change the amino acids to A49V, Q89H, Q44L, and/or Y34C. One or all the point mutations can occur on one or both alleles of the CEACAM1 gene.

Where an individual is heterozygous for one or more of these variant alleles, an antibody that binds the wild-type allele may have reduced efficacy in treating a disease or disorder associated with CEACAM1, since the antibodies can exhibit reduced affinity or ineffective binding to the variant protein.

As used herein, “affinity”, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof. The smaller the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest.

In one aspect, anti-CEACAM1 antibodies and antigen-binding fragments thereof, including the antibodies described herein by their structural characteristics, specifically bind to at least part of the binding site on CEACAM1 for one or more other members of the CEACAM family, thereby blocking CEACAM1 interactions with the one or more other members of the CEACAM family. These CEACAM family members include, but are not limited to, CEACAM3, CEACAM5, CEACAM6, and CEACAM8 (Ramani et al, Anal. Biochem. Jan. 15, 2012; 420(2); 127-38; Scheffrahn et al, J. Immunol. May 15, 2002; 168(10); 5139-46).

In one aspect, anti-CEACAM1 antibodies and antigen-binding fragments thereof, including the antibodies described herein by their structural characteristics, specifically bind to at least part of the binding site on CEACAM1 for a member of the TIM family, thereby blocking CEACAM1 interaction with the TIM family member. In some embodiments, this TIM family member is TIM-3. In some embodiments, the CEACAM1 antibody or antigen-binding fragment thereof specifically binds to one or more of CEACAM1 residues Y34, G41, N42, Q44, Q89, S93, D94, V96, and/or N97 of SEQ ID NO: 13, residues which have been indicated to be involved in CEACAM1 binding to TIM-3 (Huang et al., Nature. 2015 Jan. 15; 517(7534):386-90).

To the extent that the binding of anti-CEACAM1 antibodies to the GF-CC′ face of CEACAM1 can interfere with or disrupt interactions of the CEACAM1 polypeptide with factors that influence or mediate a CEACAM1-related disease or disorder, the antibodies can provide therapeutic benefit. Interactions of CEACAM1 with other factors associated with various pathologies are described herein for completeness.

In one aspect, anti-CEACAM1 antibodies and antigen-binding fragments thereof, including the antibodies described herein by their structural characteristics, specifically bind to at least part of the binding site on CEACAM1 for a bacterial adhesive surface protein (adhesin), thereby blocking the interaction between CEACAM1 and the adhesin. In certain embodiments, the adhesin is expressed on the surface of a CEACAM1-binding pathogenic bacterium including, but not limited to, Escherichia coli, particularly Diffusively Adhering Escherichia coli (DAEC), Neisseria gonorrhoeae, N. meningitidis, commensal Neisseria, Moraxella catarrhalis, Haemophilus influenza, Haemophilus aegyptius, Helicobacter pylori, and/or Salmonella sp.

In one embodiment, the CEACAM1 antibody or antigen-binding fragment thereof disrupts the interaction between CEACAM1 and HopQ expressed on the surface of Helicobacter pylori. In one embodiment, the CEACAM1 antibody or antigen-binding fragment specifically binds to one or more of CEACAM1 residues F29, Y34, N42, Q89, and N97, which have been predicted to be involved in CEACAM1 binding to HopQ.

In another embodiment, the CEACAM1 antibody or antigen-binding fragment thereof disrupts the interaction between CEACAM1 and an opacity-associated (Opa) adhesin protein expressed on the surface of Neisseria sp, including, but not limited to, Opa52, Opa65, Opa70, Opa72, Opa73, Opa74, and Opa75. In one embodiment, the CEACAM1 antibody or antigen-binding fragment specifically binds to one or more of CEACAM1 residues Q44 and A49, which have been predicted to be involved in CEACAM1 binding to neisserial Opa proteins.

In another embodiment, the CEACAM1 antibody or antigen-binding fragment thereof disrupts the interaction between CEACAM1 and Opa-like protein O1pA expressed on the surface of Moraxella sp.

In one embodiment, the CEACAM1 antibody or antigen-binding fragment thereof disrupts the interaction between CEACAM1 and Haemophilus influenza OMP P 1. In one embodiment, the CEACAM1 antibody or antigen-binding fragment specifically binds to one or more of CEACAM1 residues Q44 and A49, which have been predicted to be involved in CEACAM1 binding to Haemophilus influenza OMP P1.

In another embodiment, the CEACAM1 antibody or antigen-binding fragment thereof disrupts the interaction between CEACAM1 and Haemophilus aegyptius OMP P1. In one embodiment, the CEACAM1 antibody or antigen-binding fragment specifically binds to CEACAM1 residue F29, which has been predicted to be involved in CEACAM1 binding to Haemophilus aegyptius OMP P1.

In another embodiment, the CEACAM1 antibody or antigen-binding fragment thereof disrupts the interaction between CEACAM1 and C. albicans.

In another embodiment, the CEACAM1 antibody or antigen-binding fragment thereof disrupts the interaction between CEACAM1 and an influenza virus, including but not limited to H5N1.

In another embodiment, the CEACAM1 antibodies or antigen-binding fragments thereof described herein inhibit binding of CEACAM1 to a filial nematode. In one embodiment, the filial nematode is Wucheria bancrofti.

### Nucleic Acids

In some embodiments, nucleic acids encoding CEACAM1 antibodies and antigen-binding fragments thereof, as well as vectors, host cells, and expression systems including those nucleic acids, can be used to prepare or deliver anti-CEACAM1 antibodies.

The nucleic acids encoding CEACAM1 antibodies and antigen-binding fragments thereof may be, e.g., DNA, cDNA, RNA, synthetically produced DNA or RNA, or a recombinantly produced chimeric nucleic acid molecule comprising any of those polynucleotides either alone or in combination. For example, a nucleic acid can include an expression vector comprising a polynucleotide sequence encoding a CEACAM1 antibody or antigen-binding fragment thereof described herein operably linked to expression control sequences suitable for expression in a eukaryotic and/or prokaryotic host cell.

### Antibody Preparation and Expression Systems

Antibodies or antigen-binding fragments thereof as described herein are typically produced by recombinant expression. Nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells.

Once the vector has been incorporated into the appropriate host cell, the host cell is maintained under conditions suitable for high level expression of the nucleotide sequences, and the collection and purification of the cross-reacting antibodies. Expression systems for antibodies are described, for example, in PCT/US2019/065212, which is incorporated herein by reference.

### Genomic DNA Extraction and Genotyping Assays

Methods described herein include genotyping an individual with regard to their CEACAM1 genotype. Such genotyping is typically performed on a biological sample comprising genomic DNA from the subject. A number of suitable methods are available in the art and include, but are not limited to, DNA sequencing, allele specific hybridization, PCR, Allele Specific Amplification (ASA) (WO93/22456), Allele Specific Hybridization, single base extension (U.S. Pat. No. 4,656,127), ARMS-PCR, Taqman (U.S. Pat. Nos. 4,683,202; 4,683,195; and 4,965,188), oligo ligation assays, single-strand conformational analysis ((SSCP) Orita et al PNAS 86 2766-2770 (1989)), Genetic Bit Analysis (WO 92/15712) and RFLP direct sequencing, mass-spectrometry (MALDI-TOF) and DNA arrays In some embodiments, the biological sample includes cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. In some embodiments of any of the aspects, the test sample can be a bodily fluid, a buccal swab, or a tumor sample.

The above described methods may require amplification of a DNA sample from the subject, and this can be done by techniques known in the art such as PCR (see PCR Technology: Principles and Applications for DNA Amplification (ed. H. A. Erlich, Freeman Press, NY 1992; PCR Protocols: A Guide to methods and Applications (eds. Innis et al., Academic press, San Diego, Calif 1990); Mattila et al., Nucleic Acids Res. 19 4967 (1991); Eckert et al., PCR Methods and Applications 117 (1991) and U.S. Pat. No. 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self-sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods or assays as described herein. After thawing, a frozen sample can be centrifuged before being subjected to methods or assays as described herein. In some embodiments, the test sample is a clarified test sample, for example, prepared by centrifugation and collection of a supernatant. In some embodiments, a test sample can be a pre-processed test sample, for example, supernatant or filtrate resulting from a treatment selected from centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during storage or processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods and assays described herein can further comprise a step of obtaining or having obtained a test sample from a subject.

Nucleic acid samples derived from cancerous and non-cancerous cells of a subject that can be used in the methods described herein to determine the genotype of a subject can be prepared by means well known in the art.

While the most likely sample for genotyping a subject will be a blood sample, in some instances, collecting nucleic acid samples from a subject can be accomplished, for example, by surgery or aspiration. In surgical procedures where cancerous tissue is removed, surgeons often remove non-cancerous tissue and/or cell samples of the same tissue type of the cancer patient for comparison. Nucleic acid samples can be isolated from such tissue of the subject for use in the methods described herein.

### Protein Extraction and Antibody Binding Assays Affinity Determination

Where applicable, the amount of CEACAM1 expressed by a subject or in or around a subject's cancer or infected tissue can be determined by methods known in the art, including, for example, ELISA, Western blot, immunohistochemistry or other immunoassay. Such an immunoassay can be performed with an antibody that binds the GF-CC′ face of CEACAM1, or, in other embodiments, using an antibody that binds another epitope. Mass spectrometry can also be used. Standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

A level which is less than a reference level can be a level which is less by at least about 10%, at least about 20%, at least about 50%, at least about 60%, at least about 80%, at least about 90%, or less relative to the reference level. In some embodiments, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments, a level which is more than a reference level can be a level which is statistically significantly greater than the reference level.

In some embodiments, when a subject is found to be heterozygous for one or more allelic variants of CEACAM1 including A49V, Q89H, Q44L and/or Y34C, methods described herein can further include assaying a sample comprising CEACAM1 from the subject to determine the degree to which the subject's CEACAM1 is bound by an agent comprising an antibody or antigen-binding domain that binds an epitope in the GF-CC′ domain of CEACAM1. The amount and/or dosing regimen for such an agent can be adjusted as a function of the degree of binding of the agent to the subject's CEACAM1. That is, where the antibody agent is found to bind to the subject's CEACAM1 protein, but to do so with, e.g., reduced affinity, one can increase the dosage and/or frequency of dosing of the antibody agent that the subject receives to increase efficacy. Such an increased dosage can be, for example, proportional to the degree of reduced affinity determined.

Methods to measure antibody binding are known to a skilled artisan. Such methods include, for example ELISA (enzyme linked immunosorbent assay), western blot, immunoprecipitation, and immunofluorescence assays. Antibody dissociation constants and affinities can be determined, for example, by a surface plasmon resonance-based assay (such as the BIAcore assay described in PCT Application Publication No. WO2005/012359); Forte Bio Octet™ analysis, enzyme-linked immunosorbent assay (ELISA); and competition assays (e.g., RIA's), for example. An antibody or antigen-binding fragment thereof as described herein is said to be “specific for” or to “specifically bind” or “selectively bind” a first target or antigen compared to a second target or antigen when it binds to the first antigen with an affinity (as described above, and suitably expressed, for example as a K_{D} value) that is at least 1000 times, 10000 times or more better than the affinity with which said amino acid sequence or polypeptide binds to another given polypeptide. Generally, a molecule that “specifically binds,” “selectively binds” or “is specific for” a given target will bind with a K_{D} of 10⁻⁶ M (1000 nM) or lower, e.g., 10⁻⁷ M, 10⁻⁸ M, 10⁻⁹ M, 10⁻¹⁰ M, 10⁻¹¹ M, or more. Specific binding can be influenced by, for example, the affinity and avidity of the polypeptide agent and the concentration of polypeptide agent. The person of ordinary skill in the art can determine appropriate conditions under which polypeptide agents as described herein selectively bind the target using any suitable methods, such as titration of a polypeptide agent in a suitable binding assay. An ELISA can be adapted to provide a measure of antibody affinity, e.g., via a competitive ELISA. Other approaches for determining the affinity of a given antibody (e.g., an antibody that binds the GF-CC′ face of CEACAM1, e.g., a CP08H03/Vk8 S29A- or CP08H03/CP08F05-based antibody agent) are known in the art. Non-limiting examples include surface plasmon resonance (e.g., via Biacore™ assay) and biolayer interferometry (BLI), e.g., via Octet™ assay (Forte Bio)). Such assays can determine the affinity of the antibody for the CEACAM1 in a sample from a subject.

In general, if the antibody is found to bind the subject's CEACAM1 with a K_{D} higher than 1 micromolar, it would not be expected that increasing dosage or frequency of administration will be effective. In other embodiments, if the antibody is found to bind the subject's CEACAM1 with a K_{D} higher than 10⁻⁷M, it would not be expected that increasing dosage or frequency of administration will be effective. However, where the affinity is in the range of 100 nM to 1 nM, dosage can be adjusted, increasing dosage and/or frequency of administration proportionally to the degree of difference in affinity relative to binding to wild-type CEACAM by the same antibody agent. The CEACAM1 antibodies or antigen-binding fragments thereof described herein bind to CEACAM1 with a K_{D} of 10⁻⁶ to 10⁻¹² mol/l, 10⁻⁷ to 10⁻¹² mol/l, 10⁻⁸ to 10⁻¹² mol/l, 10⁻⁹ to 10⁻¹² mol/l, 10⁻¹⁰ to 10⁻¹² mol/l, or 10⁻¹¹ to 10⁻¹² mol/l.

### Methods for Modulating CEACAM1 Activity

In one aspect, anti-CEACAM1 antibodies and antigen-binding fragments thereof described herein are useful for decreasing the interaction between CEACAM1 and another member of the CEACAM family, including, but not limited to, CEACAM1, CEACAM3, CEACAM5, CEACAM6, and CEACAM8. In some embodiments, the antibody or antigen-binding fragment thereof disrupts the homophilic interaction between CEACAM1 monomers.

In another aspect, anti-CEACAM1 antibodies and antigen-binding fragments thereof described herein are useful for decreasing the interaction between CEACAM1 and a member of the TIM family, including TIM-3. In some embodiments, the antibody or antigen-binding fragment thereof disrupts the heterophilic interaction between CEACAM1 and TIM-3. Disruption of the interaction between CEACAM1 and TIM-3 by using the antibodies and antigen-binding fragments thereof contemplated by the invention may reverse CEACAM1 inhibitory functions while maintain TIM-3 activating functions.

Inhibition of CEACAM1 can be useful for reducing immunosuppression, e.g., T cell tolerance. Immunosuppression can be mediated by immune inhibitory receptors expressed on the surface of an immune cell, and their interactions with their ligands. For example, cytotoxic CD8 T cells can enter a state of “functional exhaustion,” or “unresponsiveness” whereby they express inhibitory receptors that prevent antigen-specific responses, such as proliferation and cytokine production. Accordingly, by inhibiting the activity and/or expression of such inhibitory receptors, an immune response to a cancer or tumor that is suppressed, inhibited, or unresponsive, can be enhanced or de-repressed. Such enhancements or reversal of inhibition of the immune response can lead to greater T cell activity, responsiveness, and/or ability or receptiveness with regards to activation.

Methods of measuring T cell activity and thereby tolerance or relative reduction or lack of activity are known in the art. Thus, in some embodiments, one or more of parameters or markers of T cell tolerance can be assayed to determine whether the anti-CEACAM1 antibodies or antigen-binding fragments thereof disclosed herein inhibit CEACAM1 activity. As one example, reduced levels of factors associated with tolerance can be indicative of tolerance inhibition, including, but not limited to reduced levels of IL-2. Other cytokines and factors associated with or involved in tolerance are known in the art. Reduction of T cell tolerance can also be assessed by examination of tumor infiltrating lymphocytes or T lymphocytes within lymph nodes that drain from an established tumor. Such T cells exhibit features of exhaustion through expression of cell surface molecules such as PD1, TIM-3 or LAG-3, for example, and decreased secretion of cytokines such as interferon-γ. Accordingly, evidence that T cell tolerance has been reduced in the presence of CEACAM1 antibodies or antigen-binding fragments thereof includes, e.g., increased quantities of T cells with (a) antigen specificity for tumor associated antigens (e.g., as determined by major histocompatibility complex class I or class II tetramers which contain tumor associated peptides) and (b) the secretion of high levels of interferon-γ and cytolytic effector molecules such as granzyme-B, relative to that observed in the absence of the antibody agent.

### Methods of Treatment

In one aspect, described herein are methods of determining whether a given subject's disease or disorder is likely to respond to a given CEACAM1 antibody agent. In other aspects, described herein are methods of treating a subject whose disease or disorder is found likely to respond to a CEACAM antibody agent with such an agent. In such methods, a therapeutically effective amount of an antibody or antigen-binding portion thereof is administered to a mammal in need thereof. Although antibodies or antigen-binding portions thereof as described herein are particularly useful for administration to humans, they may be administered to other mammals as well. A “therapeutically effective amount” is an amount of an antibody agent as described herein that, when administered to a mammal, is effective in producing the desired therapeutic effect.

In some aspects, the antibody or antigen-binding fragment thereof binds to CEACAM1 expressed by an exhausted T cell or natural killer (NK) cells, thereby recovering T cell and NK cell activity and leading to increased anti-tumor responses. In other aspects, the antibody or antigen-binding fragment thereof binds to CEACAM1 expressed by a tumor cell, thereby inhibiting tumor cell metastasis and the formation of a cancer stem cell niche. In yet another aspect, the antibody or antigen-binding fragment thereof binds to CEACAM1 expressed by macrophages associated with fibrosis in the tumor environment thereby inhibiting fibrosis. In another aspects, the antibody or antigen-binding fragment thereof binds to CEACAM1 expressed by other stromal cells in the tumor microenvironment such as vascular endothelium cells, thereby inhibiting angiogenesis.

As such, also provided herein are methods of treating a subject having a cancer or tumor and/or reducing tumor growth, comprising administering an effective amount of a CEACAM1-antibody agent as described herein. “Reducing” in this context includes inhibiting and/or reversing and can refer to, for example, the symptoms of the disorder being treated, the presence or size of metastases or micrometastases, the size of the primary tumor, or the presence or the size of a dormant tumor.

By “subject” is meant a mammal, including, but not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “prevent,” “prevention,” and the like refer to acting prior to overt disease or disorder onset, to prevent the disease or disorder from developing or to minimize the extent of the disease or disorder, or slow its course of development.

Embodiments described herein can be used for treating metastasis, which relates to the spreading of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Also contemplated are methods of reducing cancer stemness comprising the administration of CEACAM1 antibody agents as described herein. Cancer stemness may refer to the ability of a cell to self-renew and to generate an additional, phenotypically distinct cell type. Cancer stem cells (CSCs) are cancer cells that exhibit stem-cell like properties. CSCs often exhibit at least one hallmark of cancer, and are capable of generating at least one additional, phenotypically distinct cell type. Furthermore, cancer stem cells are capable of both asymmetric and symmetric replication. It is appreciated that a cancer stem cell may result from differentiated cancer cells that acquire stemness traits and/or stem cells that acquire phenotypes associated with cancer cells. Alternatively, cancer stem cells can reconstitute non-stromal cell types within a tumor.

CEACAM1 is expressed by many tumor types and CEACAM1 may regulate or participate in the regulation of the growth and metastatic behavior of the tumor. In another embodiment, CEACAM1 inhibition will decrease tumor growth and metastasis.

CEACAM1 expression on subsets of macrophages is associated with fibrosis during carcinogenesis. In a further embodiment, CEACAM1 inhibition will decrease tumor-associated fibrosis.

Cancers that may be treated by the compositions and methods contemplated by the invention include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated include, but are not limited to sarcomas, carcinomas, and leukemias, lymphomas and myelomas, blastomas, and brain and spinal cord cancers. Adult tumors/cancers and pediatric tumors/cancers are also included. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NEIL; intermediate grade diffuse NHL; high grade immunoblastic NEIL; high grade lymphoblastic NEIL; high grade small non-cleaved cell NHL; bulky disease NEIL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. A patient can have more than one type of cancer.

The efficacy of the treatment methods for cancer comprising therapeutic formulations of the antibodies and antigen-binding fragments thereof described herein can be measured by various endpoints commonly used in evaluating cancer treatments, including but not limited to, tumor regression, tumor weight or size shrinkage, time to progression, duration of survival, progression free survival, overall response rate, duration of response, and quality of life. In the case of cancers, the therapeutically effective amount of a CEACAM1-antibody agent can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. In cases where a patient has more than one type of cancer, the therapeutically effective amount of the CEACAM1-antibody agent is an amount effective in treating at least one of the cancers. To the extent the CEACAM1-antibody agent acts to prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

Immune checkpoint proteins interact with specific ligands that send a signal into the T cell and switch off or inhibit T cell function. By expressing high levels of checkpoint proteins on their surface, cancer cells can control the function of T cells that enter the tumor microenvironment, thus suppressing the anticancer immune response. The immune checkpoint protein Programmed Death-1 (PD-1) is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1 (Freeman et al., 2000; Latchman et al., 2001). Inhibition of the PD-1/PD-L1 interaction can promote potent antitumor activity. Examples of PD-1 inhibitors include, but are not limited to, Pembrolizumab (MK-3475), Nivolumab (MDX-1106), Cemiplimab-rwlc (REGN2810), Pidilizumab (CT-011), Spartalizumab (PDR001), tislelizumab (BGB-A317), PF-06801591, AK105, BCD-100, BI 754091, JS001, LZM009, MEDI0680, MGA012, Sym021, TSR-042. Examples of PD-L1 inhibitors include, but are not limited to, Atezolizumab (MPDL3280A), Durvalumab (MED14736), Avelumab (MSB0010718C), BGB-A333, CK-301, CS1001, FAZ053, KN035, MDX-1105, MSB2311, SHR-1316.

However, there is a significant population of cancer patients receiving checkpoint inhibitor therapy that (1) fail to respond to this type of therapy (innate or primary resistance) or that (2) initially respond but eventually develop disease progression (secondary or acquired resistance). Resistant cancer may also be referred to as refractory cancer. As shown in the PD-1/CEACAM1 data from PCT/US2019/065212, incorporated herein by reference in its entirety, tumor associated cells isolated from patients with acquired resistance to PD-1/PD-L1 inhibitors upregulate CEACAM1 expression relative to tumor associated cells isolated from naive patients, that had not been exposed to PD-1 inhibitors. When CEACAM1 is expressed in the setting of acquired resistance, the CEACAM1 bearing cells are more like likely to be effector memory rather than central memory cells, consistent with a reduction of an anti-cancer response in the resistant patients.

As such, among the methods described herein for using CEACAM1 antibody agents is the use of such agents for the treatment of patients with resistance to checkpoint inhibitors such as inhibitors of PD-1, PD-L1, and/or CTLA-4. In some embodiments, the CEACAM1 antibody used in the treatment of patients with resistance to inhibitors of PD-1, PD-L1, and/or CTLA-4 is CP08H03/Vk8 S29A or CP08H03/CP08F05 or agents including the antigen-binding domain(s) thereof. In some embodiments, the resistance is innate or primary resistance. In some embodiments, the resistance is secondary or acquired resistance. In some embodiments, the administered CEACAM1 antibody agent, reverses T cell exhaustion in patients resistant to checkpoint inhibitor therapy. Any cancer exhibiting PD-1, PDL-1 and/or CTLA-4 inhibitor resistance is suitable for treatment with such methods. In some embodiments, the CEACAM1 antibody agent is administered to a patient that has not previously received checkpoint inhibitor therapy.

In another aspect, the methods include the use of the CEACAM1 antibody agents in the treatment of patients with resistance to therapy with other checkpoint inhibitors, including but not limited to, inhibitors of PD-L2, B7-H3, B7-H4, BTLA, HVEM, GALS, LAG3, TIM-3, VISTA, KIR, 2B4 (belongs to the CD2 family of molecules and is expressed on all NK, y6, and memory CD8+ (4) T cells), CD160 (also referred to as BY55), CGEN-15049, CHK1 and CHK2 kinases, A2aR and various B-7 family ligands (including, but are not limited to, B7-1, B7-2, B7-DC, B7-H1, B7-H2, B7-H3, B7-H4, B7-H5, B7-H6 and B7-H7).

In another aspect, the methods include using the CEACAM1 antibody agents for the treatment of a subject in need of reducing and/or preventing the colonization of mammalian epithelia with Candida albicans and/or bacteria expressing bacterial adhesins (including, but not limited to, Escherichia coli, particularly Diffusively Adhering Escherichia coli (DAEC), Neisseria gonorrhoeae, N. meningitidis, commensal Neisseria, Moraxella catarrhalis, Haemophilus influenza, Haemophilus aegyptius, Helicobacter pylori, and/or Salmonella sp.). In another aspect, the methods include using the CEACAM1 antibody agents for reducing replication of an influenza virus and/or for reducing the release of pro-inflammatory cytokines or chemokines associated with an infection with an influenza virus. In some embodiments, the influenza virus is H5N1. In another aspect, the methods include using the CEACAM1 antibody agents for the treatment of a subject in need of reducing and/or preventing the infection with a filial nematode such as Wucheria bancrofti. In another aspect, the methods include using the CEACAM1 antibody agents for the treatment of a subject in need of reducing and/or preventing the development of lymphedema and/or hydrocele associated with an infection with a filial nematode such as Wucheria bancrofti. In one embodiment, the methods include using the CEACAM1 antibody agents for reducing invasion of a subject's lymphatic system with a filarial worm in a subject in need thereof. In one embodiment, the filial nematode is Wucheria bancrofti. A subject may be infected with more than one of a bacterium expressing a bacterial adhesin, Candida albicans, an influenza virus and/or a filial nematode.

In another embodiment, the methods include using the CEACAM1 antibody agents for reducing the invasion of a subject's lymphatic system with cancer cells in a subject in need thereof.

### Pharmaceutical Compositions

Where genotyping indicates that a subject's disease or disorder can be treated with an anti-CEACAM1 antibody agent (e.g., an agent that binds the GF-CC′ face of CEACAM1, including, but not limited to a CP08H03/Vk8 S29A- or CP08H03/CP08F05-based antibody agent), the antibody agent can include a therapeutically effective amount of a CEACAM1 antibody or antigen-binding fragment thereof as described herein, formulated together with one or more pharmaceutically acceptable excipients.

The dosage of active agent(s) can vary, depending on the reason for use, the individual subject, and the mode of administration. The dosage can be adjusted based on the subject's weight, the age and health of the subject, and tolerance for the compound(s) or composition. For example, depending on the disease, for an antibody or antigen-binding fragment thereof, this may require 0.1, 1.0, 3.0, 6.0, or 10.0 mg/Kg. For an IgG having a molecular mass of 150,000 g/mole (two binding sites), these doses correspond to approximately 18 nM, 180 nM, 540 nM, 1.08 μM, and 1.8 μM of binding sites for a 5 L blood volume.

The active agent and excipient(s) can be formulated into compositions and dosage forms according to methods known in the art. The pharmaceutical compositions of the present invention can be specially formulated in solid or liquid form, including those adapted for parenteral administration, for example, by subcutaneous, intratumoral, intramuscular or intravenous injection as, for example, a sterile solution or suspension.

Therapeutic compositions comprising antibodies or antigen-binding fragments thereof that bind to CEACAM1 can be formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), bulking agent, salt, surfactant and/or preservative, solvent or encapsulating material involved in carrying or transporting the therapeutic compound for administration to the subject. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents include, for example, mannitol, glycine, polyethylene glycol and sorbitol.

The use of a surfactant can reduce aggregation of a reconstituted protein and/or reduce the formation of particulates in a reconstituted formulation. The amount of surfactant added is such that it reduces aggregation of the reconstituted protein and minimizes the formation of particulates after reconstitution. Suitable surfactants include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives can be used in formulations of invention. Suitable preservatives for use in a formulation include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

The compositions comprising an antibody or antigen-binding fragment thereof and a pharmaceutically acceptable carrier can comprise the CEACAM1 antibodies or antigen-binding portions thereof at various concentrations. For example, the compositions can comprise an antibody or antigen-binding fragment thereof at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions can also comprise an antibody or antigen-binding fragment thereof at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml.

In some embodiments, the compositions comprising the antibody or antigen-binding fragment thereof and the pharmaceutically acceptable carrier are lyophilized and provided in a composition for reconstitution prior to administration.

### Methods of Administration

In some embodiments, the methods described herein relate to treating a subject having or diagnosed as having a CEACAM1 related disease or disorder with an anti-CEACAM1 antibody agent. Subjects having a CEACAM1 related disease or disorder can be identified using current methods of diagnosing a CEACAM1 related disease or disorder, including, but not limited to T cell exhaustion, altered infiltration pattern of immune cells in the microenvironment, unresponsive T cells, low levels of T cells, an increase in regulatory T cells, and/or increased expression of CEACAM1. Symptoms and/or complications of CEACAM1 related disease or disorder which characterize these conditions and aid in diagnosis are well known in the art and include but are not limited to, fatigue, weight loss, unusual swelling and/or lumps, any new or unknown reason of pain, cough, hoarseness, unusual bleeding, change in bowel or bladder habits, fever, night sweats, headaches, vision or hearing problems, or mouth changes such as sores, bleeding, pain, or numbness.

The compositions and methods described herein can be administered to a subject having or diagnosed as having a CEACAM1-related disease or disorder. In some embodiments, the methods described herein comprise administering an effective amount of compositions described herein, e.g. CEACAM1 antibody to a subject in order to alleviate a symptom of a CEACAM1-related disease or disorder. As used herein, “alleviating a symptom of a CEACAM1-related disease or disorder” is ameliorating any condition or symptom associated with the CEACAM1-related disease or disorder. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of hCEACAM1 antibody or antigen-binding fragment needed to alleviate at least one or more symptom of the disease or disorder, and relates to a sufficient amount of pharmacological composition to provide the desired effect. The term “therapeutically effective amount” therefore refers to an amount of CEACAM1 related disease or disorder that is sufficient to provide a particular anti-CEACAM1 effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of CEACAM1 antibody or antigen-binding fragment, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for CEACAM1 binding, among others. The dosage can be determined by a physician and adjusted, as necessary, to suit observed effects of the treatment.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In some embodiments, the technology described herein relates to a pharmaceutical composition comprising a CEACAM1 antibody or antigen-binding fragment as described herein, and optionally a pharmaceutically acceptable carrier. In some embodiments, the active ingredients of the pharmaceutical composition comprise a CEACAM1 antibody or antigen-binding fragment as described herein. In some embodiments, the active ingredients of the pharmaceutical composition consist essentially of a CEACAM1 antibody or antigen-binding fragment as described herein. In some embodiments, the active ingredients of the pharmaceutical composition consist of a CEACAM1 antibody or antigen-binding fragment as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a CEACAM1 antibody or antigen-binding fragment as described herein.

Therapeutic compositions comprising the antibody or antigen-binding fragment thereof can be administered in any convenient manner, including by injection, transfusion, implantation or transplantation. The compositions described herein can be administered to a patient subcutaneously, intradermally, intratumorally, intranodally, intramedullary, intramuscularly, intracranially, by intravenous or intralymphatic injection, or intraperitoneally.

In certain embodiments, the antibody or antigen-binding fragment thereof is administered to the mammal by intravenous infusion, i.e., introduction of the antibody or antigen-binding fragment thereof into a vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, a dose of a compound or a composition is administered to a subject, for example, every day, every other day, every couple of days, every third day, once a week, twice a week, three times a week, once every two weeks, or once a month. In other embodiments, two, three or four doses of a compound or a composition is administered to a subject every day, every couple of days, every third day, once a week, once every two weeks or once a month. In some embodiments, a dose(s) of a compound or a composition is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more. Duration of treatment can depend upon results or observed benefits of the treatment.

### Combination Therapies

In one aspect, the methods described herein include the administration of a CEACAM1 antibody agent that is administered with an additional therapeutic agent. Such additional agents include, but are not limited to, cytotoxic agents, chemotherapeutic agents, growth inhibitory agents, anti-inflammatory agents, anti-cancer agents, anti-neurodegenerative agents, and anti-infective agents. Agents that are used in such combination therapies can fall into one or more of the preceding categories. The administration of the antibody agent and the additional therapeutic agent can be concurrently or consecutively. The administration of the antibody or antigen-binding fragment thereof and the additional therapeutic agent can be separately or as a mixture. Further, the methods of treatment contemplated by the invention can relate to a treatment in combination with one or more cancer therapies selected from the group of antibody therapy, chemotherapy, cytokine therapy, dendritic cell therapy, gene therapy, hormone therapy, laser light therapy, and radiation therapy, among others.

Exemplary additional therapeutic agents also include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy alpha or beta particles which have a short path length. Such radionuclides kill cells to which they are in close proximity, for example neoplastic cells to which the radionuclide has attached or has entered. They have little or no effect on non-localized cells and are essentially non-immunogenic.

Exemplary additional therapeutic agents also include cytotoxic agents such as cytostatics (e.g. alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary additional therapeutic agents also include alkylating agents such as the anthracycline family of drugs (e.g. adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine 0-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary additional therapeutic agents also include transcription regulators such as actinomycin D, daunorubicin, doxorubicin, homoharringtonine, and idarubicin. Other exemplary cytostatic agents that are compatible with the methods described herein include ansamycin benzoquinones, quinonoid derivatives (e.g. quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g. carmustine, lomustine, semustine).

Exemplary additional therapeutic agents also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g. paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)); anti-angiogenesis compounds such as Angiostatin K1-3, DL-a-difluoromethyl-ornithine, endostatin, fumagillin, genistein, minocycline, staurosporine, and (±)-thalidomide.

Exemplary additional therapeutic agents also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-a, rapamycin, sex hormone-binding globulin, and thapsigargin.

Exemplary additional therapeutic agents also include enzyme inhibitors such as, S(+)-camptothecin, curcumin, (−)-deguelin, 5,6-dichlorobenz-imidazole ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879. Exemplary additional therapeutic agents also include gene regulators such as 5-aza-2^{l}-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary additional therapeutic agents also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other additional therapeutic agents that are compatible with the teachings herein include auristatins (e.g. auristatin E and monomethylauristan E), calicheamicin, gramicidin D, maytansanoids (e.g. maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

In one embodiment, the CEACAM antibody agent is administered in combination with an agent that is a checkpoint inhibitor. Such inhibitors may include small molecule inhibitors or may include antibodies, or antigen binding fragments thereof, that bind to and block or inhibit immune checkpoint receptors, or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM-3, VISTA, KIR, 2B4 (belongs to the CD2 family of molecules and is expressed on all NK, y6, and memory CD8⁺ (4) T cells), CD160 (also referred to as BY55), CGEN-15049, CHK1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM-3, GAL9, LAG3, VISTA, KIR, 2B4, CD160 and CGEN-15049. Illustrative immune checkpoint inhibitors include Tremelimumab (CTLA-4 blocking antibody), anti-0X40, and Yervoy/ipilimumab (anti-CTLA-4 checkpoint inhibitor), as well as the PD-1 and PD-L1 inhibitors described above. Checkpoint protein ligands include, but are not limited to PD-L1, PD-L2, B7-H3, B7-H4, CD28 and CD86.

In some embodiments, the CEACAM1 antibody agent is administered with a TIGIT, LAP, Podoplanin, Protein C receptor, ICOS, GITR, CD226 or CD160 inhibiting agent.

In some embodiments, the CEACAM1 antibody agent is administered with a CTLA-4, a PD-1, a PD-L1, or a PD-L2 inhibiting agent. In some embodiments, the CEACAM1 antibody agent is administered with a TIM-3 inhibiting agent.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

### EXAMPLES

### Example 1: Companion Diagnostic for Human CEACAM1 Directed Therapeutic Agents

A therapeutic monoclonal antibody candidate has been developed for human therapies (CP08; see, e.g., PCT/US2019/065212 CP08H03/Vk8 S29A- or CP08H03/CP08F05). The structural basis for binding has also been solved for this antibody to its target protein, human carcinoembryonic antigen cell adhesion molecule 1 (hCEACAM1). These studies show that this antibody (CP08) binding involves amino acids such glutamine 89 (Q89) and tyrosine 34 (Y34). These two amino acids are affected by allelic variation. This includes conversion of glutamine 89 to histidine (H) and tyrosine 34 to cysteine (C). In addition, it was observed that the alanine 49 residue is nearby to the CP08 contact site and affected by allelic variation (alanine 49 to valine (V)). CEACAM1 is distinctive in relation to other similar hCEACAM members (hCEACAM3, hCEACAM5, hCEACAM6) as it contains valine at position 49. It was predicted that the valine at position 49 would affect CP08 binding. These SNPs (A49V, Q89H, Y34C) as well as glutamine 44 to leucine (Q44L) are contained within a contiguous patch at the center of the face of hCEACAM1 where the CP08 antibody binds. Q44L may also affect binding of the drug to hCEACAM1 based upon studies with its murine parent (5F4). This suggests humans that express natural variants at these positions may bind the CP08 antibody less well and thus be less responsive (or unresponsive) to therapy with this therapeutic antibody. This hypothesis was tested by generating an A49V and Q89H double mutant of hCEACAM1.

X-ray crystallography was performed, and it was found that the A49V mutation caused a rotameric shift of phenylalanine (F) 29 residue, predicting formation of a steric clash. It was found that this double mutant (A49V,Q89H) was unable to bind CP08. This would predict that a human expressing either of these mutations or both mutations would be less responsive or unresponsive to a CP08-based therapeutic, respectively. Further, these four allelic variants were modeled on the surface of hCEACAM1 using the previously published structure of hCEACAM1. All four of these allelic variants were found to exist within a contiguous patch that lies at the center of the GFCC′ face where CP08 binds. Mutation of hCEACAM1 to an A48V/Q89H double mutant was found to abrogate binding of CP08 to hCEACAM1. Recent studies suggest that 14% of Caucasians and 43% of Africans express an A49V, Q89H haplotype which would predict that 7% and 210% of these populations, respectively, may not be responsive to the CP08-based drug if administered. In addition, Q44L and Y34C affect binding of CP08 to hCEACAM1. Further, all hCEACAM1 directed therapeutic antibodies that bind to the center of the GFCC′ face would be expected to be affected by these mutations.

There is no guidance on how to precisely apply therapeutic antibodies against hCEACAM1 in the population for clinical use in order to anticipate responsive/non-responsive populations. It has not previously been recognized that human allelic variants that characterize hCEACAM1 structure can be used as a means to determine responsiveness to anti-hCEACAM1 therapy and drugs that target the GFCC′ face of human CEACAM1, which represents the primary ligand binding site. It is expected that if an individual human harbors certain allelic variants, the binding of the anti-hCEACAM1 therapeutic antibody to its target (hCEACAM1) will be absent (homozygous A49V/Q89H haplotype) or diminished (heterozygous A49V, heterozygous Q89H or Y34C and Q44L carriers who are heterozygous or heterozygous). Knowing that the patient has or does not have one of these variants permits the selection of therapy most likely to work for the given subject. Where the subject is found to be homozygous wild-type, it is likely that a CEACAM1-related disease or disorder will be responsive to therapy with a CP08-based antibody.

Group 1 patients are those subjects who are found to be compound, homozygotes for the A49V and Q89H variants. They are considered a hCEACAM1 therapeutic “non-binder” and thus unlikely to respond to the therapeutic agent; these individuals are deemed unsuitable for therapeutic administration of a CP08-based therapy, and are also likely to be non-responders with any antibody that specifically binds the GF-CC′ face of wild-type hCEACAM1.

Group 2 patients are those subjects who are heterozygotic for A49V and/or Q89H or are found to be carriers of Y34C or Q44L of either zygosity. In this instance, these subjects are considered to be likely therapeutic candidates for administration with CP08-based antibody treatment but may require higher dosages of anti-hCEACAM1 antibody treatment for them to exhibit responses. In one embodiment, doses of CP08-based antibody agents for such patients can be adjusted on the basis of an assessment of the affinity of CP08-based antibody agents' binding to the subject's hCEACAM1 (see below).

Group 3 patients are human subjects with a homozygous wild-type configuration of the hCEACAM1 gene and thus protein. These latter subjects are considered therapeutic candidates for whom therapy with a CP08-based antibody agent is likely to be effective.

A second level of characterization can be applied to Groups 1 and 2 wherein tissue samples (blood and/or tumor) are assessed for binding to the drug. Groups 1 and 2 subjects that exhibit no evidence of binding to the therapeutic agent in these tissue sample examinations (potentially due to an absence of hCEACAM1 protein or an allelic variant unable to bind in some proportion, e.g. a low level of hCEACAM1 expression together with a particular genotypic variant) will not be considered as therapeutic candidates. Such knowledge will thus inform treatment decisions.

To define the genotypic usage of these allelic variants, a bodily fluid (e.g. blood, saliva) or tissue sample (e.g. buccal swab and/or tumor tissue) is obtained. DNA is extracted from the bodily fluid or tissue samples. To define therapeutic antibody binding, tissue samples (blood or tumor tissues) are prepared as is known in the art.

To define the genotype of the subjects, DNA from the subjects is assessed for the presence of the A49V, Q89H, Q44L and Y34C alleles and their zygosity (homozygosity or heterozygosity) determined. The methods that are used to accomplish this can include a Tagman assay, Amplicon sequencing (all four single nucleotide polymorphisms exist on a single exon that encodes a protein of 110 amino acids and thus approximately 300 bp), determination of oligonucleotide binding or other assays. Such methods are well known in the art and need not be detailed here. Individuals who are found to be compound, homozygotes for the A49V and Q89H variants (Group 1) are considered a hCEACAM1 therapeutic non-binder and thus likely to be non-responsive to the therapeutic; these individuals will be deemed unsuitable for CP08-based antibody agent therapeutic administration and no further studies will be undertaken. Such individuals benefit from not being treated with an agent not likely to provide a benefit, and thus earlier treatment with another anti-cancer agent, whether treatment with a different anti-CEACAM1 agent, one or more chemotherapeutics, immunotherapy, radiation, surgery or some combination thereof.

Human subjects who are deemed to be in Group 2 or Group 3 based upon their genotype can then be assessed for the binding of the drug to their tissues or CEACAM protein extracted therefrom. In this case, cells, for example, from peripheral blood are assessed in solution or after mounting on a fixed surface (e.g. glass slide, matrix) for solution based (flow cytometry, mass cytometry) or surface-based (immunohistochemistry [IHC] or immunofluorescence [IF]) assays to detect binding of the drug compared to a positive control that will detect hCEACAM1 protein regardless of genotype. These techniques are also well known in the art and need not be detailed here. There are many antibodies that could be used for genotype-independent binding, including commercially available antibodies that recognize hCEACAM1 domains other than the IgV domain for example. Tumor tissues can either be mechanically dissociated for solution-based assays as above or mounted in a matrix (e.g. OCT, formalin/paraffin) that allows for therapeutic antibody binding compared to control by IHC or IF. Such assays can be quantified to determine a cut-off for binding of the therapeutic antibody compared to control antibody using tissue from genotypically characterized samples that express hCEACAM1 as a control, cell systems that are transfected with genotypically-defined proteins that express the wild-type and allelic variants (e.g. human embryonic kidney 293T cells, Hela cells) or cell lines that express wild-type hCEACAM1 and or the genotypic variants generated by Crispr-Cas9 mediated mutation or other methods. Such transfected or manipulated cell lines can be characterized for their binding to the therapeutic antibody (compared to control antibody) by solution- or matrix-based approaches to establish the lower-limit of detection expected using specific quantities of the therapeutic or control antibodies.

In an alternative approach, proteins can be extracted from the tissues and assessed for binding to the therapeutic antibody relative to control using ELISA, immunoblotting, surface plasmon resonance, biolayer interferometry or similar types of immunobinding assays compared to the signals detected with fixed concentrations of wild-type or mutated proteins which in the latter case include the various genotypic combinations.

Group 2 and 3 subjects that lack binding to the drug and/or control (due to lack of hCEACAM1 expression or a level of hCEACAM1 expression in association with a particular genotype that exhibits lower binding) or those that express a particular level of binding to the drug below a certain limit are not considered therapeutic candidates for treatment.

Accordingly, a method of determining whether a patient will be responsive or non-responsive to anti-hCEACAM1 therapeutic antibody administration is disclosed. In this method, an individual who is being considered for treatment with an anti-human CEACAM1 therapeutic is examined for the expression of wild-type or mutant (allelic variants) of hCEACAM1 (A49V, Q89H, Q44L and Y34C alleles). Individuals who are compound, homozygotes for the A49V and Q89H variants are considered hCEACAM1 therapeutic “non-binders and unlikely to respond to the therapeutic agent. Individuals who are heterozygotic for the A49V and/or the Q89H variants or are found to be carriers of Y34C or Q44L of either zygosity are possible binders and may respond to the therapeutic agent if administered in an appropriate dosage. Individuals who have the wild type configuration of the hCEACAM1 gene, i.e., the gene lacks the A49V, Q89H, Q44L and Y34C variant alleles and thus the hCEACAM1 protein is the wild type, will respond to anti-hCEACAM1 monoclonal antibody therapy.

Anti-CEACAM1 antibodies and anti-CEACAM1 monoclonal antibodies are described in U.S. Pat. No. 9,556,271, WO2013/082366 and WO2020/118295, the latter of which describes CP08. Additional anti-CEACAM1 antibodies that might be used in the alternative, e.g., for subjects for whom CP08-based Abs won't work. are described in US2016/0376374 and US2020/0277398. Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

1. A method comprising:
i) receiving results of an hCEACAM1 genotyping assay performed on a sample from a subject in need of therapy for a CEACAM1-related disease or disorder; and
ii) selecting therapy with an agent comprising an antibody or antigen-binding domain that binds an epitope in the GF-CC′ domain of CEACAM1 when the subject's hCEACAM1 genotype is homozygous wild-type with regard to A49, Q89, Q44 and Y34 or heterozygous for A49V, Q89H, Q44L and/or Y34C, and
selecting therapy with an agent other than an agent comprising an antibody or antigen-binding domain that binds an epitope in the GF-CC′ domain of CEACAM1 when the subject's hCEACAM1 genotype is homozygous for A49V, Q89H, Q44L, and/or Y34C hCEACAM1 alleles.

2. A method comprising:
i) genotyping a sample from a subject in need of therapy for a CEACAM1-related disease or disorder to detect the subject's hCEACAM1 genotype; and
ii) selecting therapy with an agent comprising an antibody or antigen-binding domain that binds an epitope in the GF-CC′ domain of CEACAM1 when the subject's hCEACAM1 genotype is homozygous wild-type with regard to A49, Q89, Q44 and Y34 or heterozygous for A49V, Q89H, Q44L and/or Y34C, and
selecting therapy with an agent other than an agent comprising an antibody or antigen-binding domain that binds an epitope in the GF-CC′ domain of CEACAM1 when the subject's hCEACAM1 genotype is homozygous for A49V, Q89H, Q44L, and/or Y34C hCEACAM1 alleles.

3. The method of claim 1 or claim 2, wherein the subject has or has been diagnosed with a chronic disease.

4. The method of claim 4, wherein the chronic disease is selected from the group consisting of: cancer; a persistent infection; and a chronic viral infection.

5. The method of any one of claims 1-5, wherein the subject is a human.

6. The method of any one of claims 1-5, wherein the antibody or antigen-binding domain that binds an epitope of the GF-CC′ domain of CEACAM1 comprises a heavy chain variable region and a light chain variable region;
wherein each of the heavy chain and the light chain variable regions comprises a CDR1, CDR2, and CDR3; and
wherein:
the sequence of CDR1H comprises the sequence X₁HX₂X₃S (SEQ ID NO: 1);
wherein X₁ is A, D, N, or S;
wherein X₂ is A or G; and
wherein X₃ is an amino acid with a hydrophobic side chain including I or M; the sequence of CDR2H comprises the sequence TISSGGTYTYYPDSVKG (SEQ ID NO:2);
the sequence of CDR3H comprises the sequence HX₄X₅DYX₆PX₇WFAX₈ (SEQ ID NO:3);
wherein X₄ is D, G, or P;
wherein X₅ is F or P;
wherein X₆ is D or F;
wherein X₇ is A or Y; and
wherein X₈ is L, H, or F;
the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX₉X₁₀X₁₁X₁₂PX₁₃T (SEQ ID NO:6); wherein X₉ is W or N;
wherein X₁₀ is S or T;
wherein X₁₁ is A or an amino acid with a neutral hydrophilic side chain including S, N, and T;
wherein X₁₂ is L, F, or N; and
wherein X₁₃ is P or F.

7. The method of any one of claims 1-5, wherein the sample is selected from a blood sample, a tissue sample, a tumor sample, and a cell sample.

8. The method of any one of claims 1-5, wherein the genotyping assay comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

9. The method of any one of claims 1-5, further comprising administering the selected therapy.

10. The method of any one of claims 1-9, further comprising, when the CEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and adjusting amount and/or dosing of the agent to be administered as a function of the degree of binding of the agent to the subject's CEACAM1.

11. The method of claim 10, where the assaying comprises a method selected from flow cytometry, mass cytometry, immunohistochemistry, immunofluorescence or FRET assay, surface plasmon resonance, radioimmunoassay, and bio-layer interferometry.

12. The method of claim 2, further comprising, before the genotyping, the step of extracting genomic DNA from a sample obtained from the subject.

13. A method of treating a subject in need thereof for a CEACAM1-related disease or disorder, the method comprising:
i) genotyping a sample from the subject to detect the subject's hCEACAM1 genotype; and
ii) when the subject's hCEACAM1 genotype is homozygous wild-type with regard to A49, Q89, Q44 and Y34, administering a therapeutically effective amount of an agent comprising an antibody or antigen-binding domain that binds an epitope in the GF-CC′ domain of CEACAM1; and
iii) when the subject's hCEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and administering a therapeutically effective amount of the agent, wherein the amount or dosing schedule of the agent administered is adjusted as a function of the degree of binding of the agent to the subject's CEACAM1; and
iv) when the subject's hCEACAM1 genotype is homozygous for A49V, Q89H, Q44L and/or Y34C, administering a therapeutically effective amount of a therapy with an agent other than one comprising an antibody or antigen-binding domain that binds an epitope in the GF-CC′ domain of CEACAM1,
whereby the CEACAM1-related disease or disorder is treated.

14. The method of claim 13, wherein the subject has or has been diagnosed with a chronic disease.

15. The method of claim 14, wherein the chronic disease is selected from the group consisting of: cancer; a persistent infection; and a chronic viral infection.

16. The method of any one of claims 13-15, wherein the subject is a human.

17. The method of any one of claims 13-16, wherein the antibody or antigen-binding domain that binds an epitope of the GF-CC′ domain of CEACAM1 comprises a heavy chain variable region and a light chain variable region;
wherein each of the heavy chain and the light chain variable regions comprises a CDR1, CDR2, and CDR3; and
wherein:
the sequence of CDR1H comprises the sequence X₁HX₂X₃S (SEQ ID NO: 1);
wherein X₁ is A, D, N, or S;
wherein X₂ is A or G; and
wherein X₃ is an amino acid with a hydrophobic side chain including I or M; the sequence of CDR2H comprises the sequence TISSGGTYTYYPDSVKG (SEQ ID NO:2);
the sequence of CDR3H comprises the sequence HX₄X₅DYX₆PX₇WFAX₈ (SEQ ID NO:3);
wherein X₄ is D, G, or P;
wherein X₅ is F or P;
wherein X₆ is D or F;
wherein X₇ is A or Y; and
wherein X₈ is L, H, or F;
the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX₉X₁₀X₁₁X₁₂PX₁₃T (SEQ ID NO:6); wherein X₉ is W or N;
wherein X₁₀ is S or T;
wherein X₁₁ is A or an amino acid with a neutral hydrophilic side chain including S, N, and T;
wherein X₁₂ is L, F, or N; and
wherein X₁₃ is P or F.

18. The method of any one of claims 13-17, wherein the sample is selected from a blood sample, a tissue sample, a tumor sample and a cell sample.

19. The method of any one of claims 13-18, wherein the genotyping assay comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

20. The method of any one of claims 13-19, wherein assaying a sample from the subject comprising CEACAM1 to determine the degree to which the subject's CEACAM1 is bound by the agent comprises a method selected from flow cytometry, mass cytometry, immunohistochemistry, immunofluorescence or FRET assay, surface plasmon resonance, radioimmunoassay, and bio-layer interferometry.

21. An assay comprising:
i) extracting genomic DNA from a biological sample from a subject in need of therapy for a CEACAM1-related disease or disorder;
ii) genotyping the subject's DNA with regard to sequence corresponding to CEACAM A49, Q89, Q44 and Y34 to thereby determine whether the subject is homozygous wild-type for A49, Q89, Q44 and Y34 or homozygous or heterozygous for each of A49V, Q89H, Q44L and Y34C.

22. The assay of claim 21, wherein the CEACAM1-related disease or disorder is selected from the group consisting of: cancer; a persistent infection; and a chronic viral infection.

23. The assay of claim 21 or 22, wherein the subject is a human.

24. The assay of any one of claims 21-23, wherein the biological sample is selected from a blood sample, a tissue sample, a tumor sample, and a cell sample.

25. The assay of any one of claims 21-24, wherein the genotyping comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

26. The assay of any one of claims 21-25, further comprising
iii) assaying the subject's CEACAM1 protein to determine the degree to which the subject's CEACAM1 is bound by an antibody or antigen-binding domain that binds an epitope in the GF-CC′ domain of CEACAM1.

27. The assay of claim 27, wherein the assaying of step (iii) comprises a method selected from flow cytometry, mass cytometry, immunohistochemistry, immunofluorescence or FRET assay, surface plasmon resonance, radioimmunoassay, and bio-layer interferometry.

28. An assay comprising:
i) extracting proteins comprising CEACAM1 from a biological sample from a subject in need of therapy for a CEACAM1-related disease or disorder; and
ii) assaying the subject's CEACAM1 in the extracted proteins to determine the degree to which the subject's CEACAM1 is bound by an antibody or antigen-binding domain that binds an epitope in the GF-CC′ domain of CEACAM1.

29. The assay of claim 28, wherein the assaying of step (ii) comprises a method selected from flow cytometry, mass cytometry, immunohistochemistry, immunofluorescence or FRET assay, surface plasmon resonance, radioimmunoassay, and bio-layer interferometry.

30. The assay of claim 28 or 29, further comprising
iii) genotyping the subject's DNA with regard to sequence corresponding to CEACAM A49, Q89, Q44 and Y34 to thereby determine whether the subject is homozygous wild-type for A49, Q89, Q44 and Y34 or homozygous or heterozygous for each of A49V, Q89H, Q44L and Y34C.

31. The assay of claim 30, wherein the genotyping comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

32. A method of selecting a hCEACAM1 antibody therapy for a subject in need thereof, comprising:
analyzing genomic DNA from a biological sample taken from the subject for the subject's genotype for alleles of hCEACAM1 at sites corresponding to A49, Q89, Q44 and Y34, and
selecting therapy with an agent comprising an hCEACAM1 antibody or antigen-binding fragment thereof that binds an epitope in the GF-CC′ domain of CEACAM1 when the genotype is found to be homozygous for A49, Q89, Q44 and Y34, or heterozygous for one or more of A49V, Q89H, Q44L, and Y34C alleles for CEACAM1; or
selecting therapy with an agent other than the agent comprising an hCEACAM1 antibody or antigen-binding fragment thereof that binds an epitope in the GF-CC′ domain of CEACAM1 when the genotype is found to be homozygous for one or more of A49V, Q89H, Q44L, and Y34C alleles for CEACAM1.

33. The method of claim 32, wherein the subject has or has been diagnosed with a chronic disease.

34. The method of claim 33, wherein the chronic disease is selected from the group consisting of: cancer; a persistent infection; and a chronic viral infection.

35. The method of any one of claims 32-34, wherein the subject is a human.

36. The method of any one of claims 32-35, wherein the antibody or antigen-binding domain that binds an epitope of the GF-CC′ domain of CEACAM1 comprises a heavy chain variable region and a light chain variable region;
wherein each of the heavy chain and the light chain variable regions comprises a CDR1, CDR2, and CDR3; and
wherein:
the sequence of CDR1H comprises the sequence X₁HX₂X₃S (SEQ ID NO: 1);
wherein X₁ is A, D, N, or S;
wherein X₂ is A or G; and
wherein X₃ is an amino acid with a hydrophobic side chain including I or M; the sequence of CDR2H comprises the sequence TISSGGTYTYYPDSVKG (SEQ ID NO:2);
the sequence of CDR3H comprises the sequence HX₄X₅DYX₆PX₇WFAX₈ (SEQ ID NO:3);
wherein X₄ is D, G, or P;
wherein X₅ is F or P;
wherein X₆ is D or F;
wherein X₇ is A or Y; and
wherein X₈ is L, H, or F;
the sequence of CDR1L comprises the sequence RANSAVSYMY (SEQ ID NO:4); the sequence of CDR2L comprises the sequence LTSNRAT (SEQ ID NO:5); and the sequence of CDR3L comprises the sequence QQX₉X₁₀X₁₁X₁₂PX₁₃T (SEQ ID NO:6); wherein X₉ is W or N;
wherein X₁₀ is S or T;
wherein X₁₁ is A or an amino acid with a neutral hydrophilic side chain including S, N, and T;
wherein X₁₂ is L, F, or N; and
wherein X₁₃ is P or F.

37. The method of any one of claims 32-36, wherein the sample is selected from a blood sample, a tissue sample, a tumor sample and a cell sample.

38. The method of any one of claims 32-37, wherein the genotype analysis comprises direct probing, allele specific hybridization, PCR, Allele Specific Amplification (ASA), single base extension, ARMS-PCR, Taqman, oligo ligation assays, single-strand conformational analysis (SSCP), Genetic Bit Analysis and RFLP direct sequencing, mass-spectrometry (MALDI-TOF), and DNA arrays.

39. The method of any one of claims 32-38, further comprising administering the selected therapy.

40. The method of any one of claims 32-39, further comprising, when the CEACAM1 genotype is heterozygous for A49V, Q89H, Q44L and/or Y34C, assaying a sample from the subject comprising CEACAM1 to determine the degree to which the subject's CEACAM1 is bound by the agent comprising an antibody or antigen-binding domain that binds an epitope in the GF-CC′ domain of CEACAM1, and adjusting amount and/or dosing of the agent to be administered as a function of the degree of binding of the agent to the subject's CEACAM1.

41. The method of claim 40, wherein the assaying comprises a method selected from flow cytometry, mass cytometry, immunohistochemistry, immunofluorescence or FRETassay, surface plasmon resonance, radioimmunoassay, and bio-layer interferometry.


SEQUENCE LISTING (15 sequences)

SEQ ID NO: 1 - skipped

SEQ ID NO: 2 - protein, 17 aa, synthetic construct
  REGION 1..17: note = CDR2H
TISSGGTYTYYPDSVKG

SEQ ID NO: 3 - protein, 12 aa, synthetic construct
  REGION 1..12: note = CDR3H
  VAR_SEQ 2: note = X is D, G or P
  VAR_SEQ 3: note = X is F or P
  VAR_SEQ 6: note = X is D or F
  VAR_SEQ 8: note = X is A or Y
  VAR_SEQ 12: note = X is L, H, or F
HXXDYXPXWFAX

SEQ ID NO: 4 - protein, 10 aa, synthetic construct
  REGION 1..10: note = CDR1L
RANSAVSYMY

SEQ ID NO: 5 - protein, 7 aa, synthetic construct
  REGION 1..7: note = CDR2L
LTSNRAT

SEQ ID NO: 6 - protein, 9 aa, synthetic construct
  VAR_SEQ 3: note = X is W or N
  VAR_SEQ 4: note = X is S or T
  VAR_SEQ 5: note = X is A or amino acid with neutral hydrophilic side chain S, N or T
  VAR_SEQ 6: note = X is L, F, or N
  VAR_SEQ 8: note = X is P or F
QQXXXXPXT

SEQ ID NO: 7 - protein, 5 aa, synthetic construct
  REGION 1..5: note = CDR1H
SHGMS

SEQ ID NO: 8 - protein, 12 aa, synthetic construct
  REGION 1..12: note = CDR3H
HDFDYFPYWFAH

SEQ ID NO: 9 - protein, 9 aa, synthetic construct
  REGION 1..9: note = CDR3L
QQWSSNPPT

SEQ ID NO: 10 - protein, 9 aa, synthetic construct
  REGION 1..9: note = CDR3L
QQWTSNPPT

SEQ ID NO: 11 - protein, 121 aa, synthetic construct
  REGION 1..121: note = heavy chain variable region
EVQLLESGGGLVQPGGSLRLSCAASGFIFSSHGMSWVRQAPGKGLEWVATISSGGTYTYYPDSVKGRFTISRDNSKNTLYLQMNSLKAEDTAMYYCARHDFDYFPYWFAHWGQGTLVTVSS

SEQ ID NO: 12 - protein, 106 aa, synthetic construct
  REGION 1..106: note = light chain variable region
EIVLTQSPATLSLSPGERATLSCRANSAVSYMYWYQQKPGQAPRPWIYLTSNRATGVPARFSGSGSGTDYTLTISSLEPEDFAVYYCQQWSSNPPTFGQGTKLEIK

SEQ ID NO: 13 - protein, 492 aa, synthetic construct
  REGION 1..492: note = The mature form of CEACAM1 (without signal sequence)
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 14 - protein, 10 aa, synthetic construct
  REGION 1..10: note = CDR1 of the light chain variable region
RANSSVSYMY

SEQ ID NO: 15 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ